(19) United States
(12) Patent Application Publication
(10) Pub. No.: US 2023/0083178 A1
(43) Pub. Date: Mar. 16, 2023

(54) MEASUREMENT APPARATUS

(71) Applicant: Panasonic Intellectual Property Management Co., Ltd., Osaka (JP)

(72) Inventors: TAKAMASA ANDO, Osaka (JP); SHUNSUKE IMAI, Osaka (JP); HITOSHI TOMINAGA, Kyoto (JP)

(21) Appl. No.: 18/057,758

(22) Filed: Nov. 21, 2022

Related U.S. Application Data
(63) Continuation of application No. PCT/JP2021/019291, filed on May 21, 2021.

(30) Foreign Application Priority Data
Jun. 15, 2020 (JP) 2020-102941

(51) Int. Cl. G01S 7/4865; G01S 7/481; G01S 17/10; G01S 7/4861

(52) U.S. Cl. CPC G01S 7/4865; G01S 7/4815; G01S 7/4811; G01S 17/10; G01S 7/4861

(57) ABSTRACT

A measurement apparatus includes first and second light sources, a diffuser, a photodetector, and a processing circuit. The first and second light sources respectively output first emitted light and second emitted light. The mirror causes the first emitted light and the second emitted light to be concentrated and incident on one region of the diffuser. The photodetector detects first reflected light and second reflected light that emanate from a subject due to the first emitted light and the second emitted light diffused by the diffuser, respectively. The processing circuit generates and outputs information related to the subject, based on the result of detection of the first reflected light and the second reflected light by the photodetector. The distance between two light spots respectively formed on the one region by the first emitted light and the second emitted light is less than the distance between the first and second light sources.

### BACKGROUND

### 1. Technical Field

The present disclosure relates to a measurement apparatus.

### 2. Description of the Related Art

Techniques exist that irradiate a subject such as a living body with light, and detect light that has passed through the interior of the subject to thereby acquire internal information on the subject. For example, Japanese Unexamined Patent Application Publication No. 2017-202328 discloses an apparatus that acquires internal information on a subject by removing noise resulting from the surface reflection component of light reflected from the subject. International Publication No. 2016/006027 discloses an apparatus that detects pulse waves from an image acquired with a camera. The techniques mentioned above employ a measurement apparatus such as a camera to achieve non-contact biometric measurements.

### SUMMARY

With such a measurement apparatus designed to acquire internal information on a subject in a non-contact manner, motion of the subject or motion of the measurement apparatus during measurement causes an error in a signal acquired by the measurement apparatus.

One non-limiting and exemplary embodiment provides a measurement technique that makes it possible to reduce an error in a signal that occurs due to motion of the subject or motion of the measurement apparatus during measurement.

In one general aspect, the techniques disclosed here feature a measurement apparatus including a first light source, a second light source, a diffuser, a mirror, a photodetector, and a processing circuit. The first light source emits first emitted light. The second light source emits second emitted light. The mirror causes the first emitted light and the second emitted light to be concentrated and incident on one region of the diffuser, by changing a direction of propagation of at least one selected from the group consisting of the first emitted light and the second emitted light. The photodetector detects first reflected light, and second reflected light. The first emanates from a subject due to the first emitted light diffused by the diffuser. The second reflected light emanates from the subject due to the second emitted light diffused by the diffuser. The processing circuit generates and outputs information related to the subject, based on a result of detection of the first reflected light and the second reflected light by the photodetector. The first emitted light forms a first light spot on the one region of the diffuser. The second emitted light forms a second light spot on the one region of the diffuser. A distance between the first light spot and the second light spot is less than a distance between the first light source and the second light source.

The technique according to the present disclosure makes it possible to reduce an error in a signal that occurs due to motion of the subject or motion of the measurement apparatus during measurement.

It should be noted that general or specific aspects of the present disclosure may be implemented as a system, an apparatus, a device, a method, an integrated circuit, a computer program, a storage medium such as a computer-readable storage disk, or any selective combination thereof. Examples of computer-readable storage media may include non-volatile storage media such as a Compact Disc-Read Only Memory (CD-ROM). The apparatus or device may be made up of one or more apparatuses or devices. If the apparatus or device is made up of two or more apparatuses or devices, the two or more apparatuses or devices may be disposed in a single piece of equipment, or may be disposed separately in two or more discrete pieces of equipment. As used herein and in the claims, the term “apparatus” or “device” may mean not only a single apparatus or device but also a system including multiple apparatuses or devices.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 schematically illustrates a measurement apparatus according to an exemplary embodiment of the present disclosure;

FIG. 2 illustrates an exemplary configuration of a photodetector;

FIG. 3A schematically illustrates, for a case where a light pulse Iₚ has an impulse waveform, exemplary time variations of a surface reflection component I₁ and an internal scattering component I₂ that are contained in a reflected light pulse;

FIG. 3B schematically illustrates, for a case where a light pulse Iₚ has a rectangular waveform, exemplary time variations of a surface reflection component I₁ and an internal scattering component I₂ that are contained in a reflected light pulse;

FIG. 4 is a flowchart illustrating an overview of how a control circuit operates in controlling a first light source, a second light source, and a photodetector;

FIG. 5A illustrates an exemplary configuration of a diffuser, light sources, and an optical system;

FIG. 5B illustrates exemplary illuminance distributions for a region represented by dashed lines in FIG. 5A;

FIG. 5C schematically illustrates an example of two adjacent light spots formed on one region of a diffuser;

FIG. 5D is a graph representing, with a human forehead as a target portion, the relationship between the inclination angle of the forehead and the tolerance for the distance on a diffuser between two light spots formed by different wavelengths of light;

FIG. 6 illustrates another exemplary configuration of a diffuser, light sources, and an optical system;

FIG. 7 illustrates an exemplary configuration of a light emitter including multiple light-emitting elements integrated into a single package;

FIG. 8 illustrates another exemplary configuration of light emitters;

FIG. 9 schematically illustrates how rays of light diffused by the diffuser are incident on the target portion;

FIG. 10 is a graph illustrating the incident-angle dependence of the diffuse reflectance of the target portion according to the configuration illustrated in FIG. 9;

FIG. 11 is a flowchart illustrating an exemplary method for generating calibration data;

FIG. 12 is a flowchart illustrating an exemplary method for signal correction that is executed during measurement by use of calibration data; and

FIG. 13 illustrates another exemplary configuration of a diffuser, light sources, and an optical system.

### DETAILED DESCRIPTIONS

Embodiments described below each represent a generic or specific example. Specific details set forth in the following description of embodiments, such as numeric values, shapes, materials, components, the placement and connection of components, steps, and the order of steps, are for illustrative purposes only and not intended to limit the technique according to the present disclosure. Those components in the following description of embodiments which are not cited in the independent claim representing the most generic concept of the present disclosure will be described as optional components. The figures are schematic and not necessarily to exact scale. Further, in the figures, the same reference signs are used to designate the same or similar components. Repetitive descriptions will be omitted or simplified in some cases.

According to the present disclosure, each circuit, unit, apparatus, device, component, or part, or each functional unit in block diagrams may, in whole or in part, be implemented by one or more electronic circuits including, for example, a semiconductor device, a semiconductor integrated circuit (IC), or a large scale integration (LSI). An LSI or an IC may be integrated in a single chip or may be a combination of multiple chips. For example, functional blocks other than a memory element may be integrated in a single chip. Although herein called LSI or IC, such electronic circuit is called differently depending on the degree of integration, and may be an electronic circuit called a system LSI, a very large scale integration (VLSI), or ultra large scale integration (ULSI). A field programmable gate array (FPGA), which is programmed after manufacture of an LSI, or a reconfigurable logic device, which allows reconfiguration of connections inside an LSI or allows set-up of circuit segments inside an LSI, may be used for the same purpose.

Further, the functions or operations of circuits, units, apparatuses, devices, components, or parts may, in whole or in part, be implemented by software processing. In this case, the software is stored in one or more non-transitory storage media such as ROMs, optical disks, or hard disk drives, and when the software is executed by a processor, functions specified in the software are executed by the processor and peripheral devices. A system, or apparatus or device may include one or more non-transitory storage media in which the software is stored, a processor, and a required hardware device, such as an interface.

First, an overview of embodiments of the present disclosure will be described below.

A measurement apparatus according to one aspect of the present disclosure includes a first light source, a second light source, a diffuser, a mirror, a photodetector, and a processing circuit. The first light source emits first emitted light. The second light source emits second emitted light. The mirror causes the first emitted light and the second emitted light to be concentrated and incident on one region of the diffuser, by changing a direction of propagation of at least one selected from the group consisting of the first emitted light and the second emitted light. The photodetector detects first reflected light, and second reflected light. The first reflected light emanates from a subject due to the first emitted light diffused by the diffuser. The second reflected light emanates from the subject due to the second emitted light diffused by the diffuser. The processing circuit generates and outputs information related to the subject, based on a result of detection of the first reflected light and the second reflected light by the photodetector. The first emitted light forms a first light spot on the one region of the diffuser. The second emitted light forms a second light spot on the one region of the diffuser. A distance between the first light spot and the second light spot is less than a distance between the first light source and the second light source. The measurement apparatus according to one aspect of the present disclosure may include multiple mirrors.

The photodetector may detect the first reflected light and the second reflected light individually. Alternatively, the photodetector may detect either one of the following types of light: light generated by superposition of the first reflected light and the second reflected light; and reflected light generated due to light generated by superposition of the first reflected light and the second reflected light. The expression “result of detection of the first reflected light and the second reflected light” may include a result represented by a signal generated in response to the photodetector detecting each of the first reflected light and the second reflected light. Alternatively, the expression may include a detection result represented by a signal generated in response to the photodetector detecting either one of the following types of light: reflected light generated due to light generated by superposition of the first reflected light and the second reflected light; and light generated by superposition of the first reflected light and the second reflected light.

The expression “distance between the first light spot and the second light spot” means the distance between the center or center of gravity of the first light spot and the center or center of gravity of the second light spot. The expression “distance between the first light source and the second light source” means the distance between the center of the first light source and the center of the second light source. As described above, when reference is made herein to the distance between two adjacent light spots or two adjacent light sources, this means the distance between their centers or centers of gravity.

According to the configuration mentioned above, one or more mirrors are placed so that the first emitted light emitted from the first light source, and the second emitted light emitted from the second light source are allowed to be concentrated and incident on a relatively narrow region of the diffuser. As a result, diffuse light due to the first emitted light, and diffuse light due to the second emitted light are incident on the target portion of the subject at substantially the same incident angle. This helps to ensure that even if the target portion and the measurement apparatus change in their relative positions due to motion of the subject or motion of the measurement apparatus, a change in illuminance or reflectance on the target portion does not depend on the difference in quantity of light or illuminance non-uniformity between the light sources. This effectively facilitates correction, that is, calibration of a signal acquired by the photodetector.

A measurement apparatus according to another aspect of the present disclosure includes a first light emitter, a diffuser, a photodetector, and a processing circuit. The first light emitter includes a first light source that emits first emitted light, a second light source that is adjacent to the first light source and emits second emitted light, and a first sub-mount that supports the first light source and the second light source. The diffuser is disposed in an optical path of each of the first emitted light and the second emitted light. The photodetector detects first reflected light, and second reflected light. The first reflected light emanates from a subject due to the first emitted light diffused by the diffuser. The second reflected light emanates from the subject due to the second emitted light diffused by the diffuser. The processing circuit generates and outputs information related to the subject, based on a result of detection of the first reflected light and the second reflected light by the photodetector.

According to the configuration mentioned above, the first light source and the second light source are disposed on the same sub-mount. The first light source and the second light source are integrated as a single package. As with the previously mentioned configuration, this configuration ensures that the first emitted light emitted from the first light source, and the second emitted light emitted from the second light source are concentrated and incident on a relatively narrow region of the diffuser. This makes it possible to provide the same effect as that of the previously mentioned aspect.

The distance on the diffuser between the center of the first light spot and the center of the second light spot may be, for example, less than or equal to 5 mm. A distance of less than or equal to 5 mm between two adjacent light spots is herein interpreted to mean that their positions are substantially the same. The distance between the respective centers of the two light spots may be less than or equal to 2 mm.

It is assumed that the first light spot and the second light spot respectively have widths w1 and w2 on the diffuser along their major axes. The distance between the first light spot and the second light spot can be made, for example, less than w1 and w2. The light sources and the optical system may be positioned to allow the first emitted light and the second emitted light to be incident on the diffuser in at least partially overlapping relation to each other.

The measurement apparatus may further include a first collimator lens disposed in an optical path between the first light source and the diffuser, and a second collimator lens disposed in an optical path between the second light source and the diffuser. The presence of a collimator lens in the optical path between each light source and the diffuser makes it possible to collimate emitted rays of light. This allows collimated light to be concentrated and incident on one region of the diffuser. This makes it possible to further reduce the influence of the difference in intensity between the light sources on the variation of illuminance on the target portion associated with motion of the subject or motion of the measurement apparatus.

The first emitted light may be incident on the diffuser at an incident angle equal to an incident angle at which the second emitted light is incident on the diffuser. That is, the light sources and the optical system may be positioned to allow the first emitted light and the second emitted light to be incident on the diffuser at the same incident angle. This configuration makes it possible to further reduce the influence of the difference in intensity between the light sources on the variation of illuminance on the target portion associated with motion of the subject or motion of the measurement apparatus. The expression “same incident angle” as used herein means substantially the same incident angle, and does not necessarily mean strictly the same incident angle.

The diffuser may have, for example, depressions or projections on its surface. Such a diffuser diffuses incident rays in diverse random directions, which helps to reduce illuminance non-uniformity on the target portion.

The first emitted light may have a wavelength of, for example, greater than or equal to 650 nm and less than 805 nm. The second emitted light may have a wavelength of, for example, greater than or equal to 805 nm and less than or equal to 950 nm. As will be described later, these wavelengths are suited for, for example, applications involving measurement of the concentrations of oxygenated hemoglobin and deoxygenated hemoglobin in the blood within a living body.

The first emitted light may have a wavelength equal to a wavelength of the second emitted light. When it is stated herein that two wavelengths are “equal”, this means that these wavelengths are substantially the same, and does not necessarily mean that these wavelengths are strictly the same. Such emission of the same wavelength of light from multiple light sources makes it possible to enhance the intensity of the wavelength of light. This helps to, for example, compensate for shortage of light quantity even if each individual light source provides only a small quantity of light.

The measurement apparatus may further include a control circuit that controls the first light source, the second light source, and the photodetector. The first reflected light and the second reflected light may be pulsed light. The control circuit may cause the first light source to emit the first emitted light, and cause the second light source to emit the second emitted light. The control circuit may cause the photodetector to detect a first component of the first reflected light in a first falling period, which is a period from when a decrease in intensity of the first emitted light begins to when the decrease ends. The control circuit may cause the photodetector to detect a second component of the second reflected light in a second falling period, which is a period from when a decrease in intensity of the second emitted light begins to when the decrease ends. The processing circuit may generate the information, based on an intensity of the first component of the first reflected light detected by the photodetector and an intensity of the second component of the second reflected light detected by the photodetector. This configuration helps to ensure that, for example, if the subject is a living body, information related to blood flow in a relatively deep part of the living body can be acquired as biometric information.

The measurement apparatus may further include a third light source that emits third emitted light. The photodetector may further detect third reflected light emanating from the subject due to the third emitted light diffused by the diffuser. The processing circuit may generate and output the information, based on a result of detection of the first reflected light, the second reflected light, and the third reflected light by the photodetector.

The photodetector may detect the first reflected light, the second reflected light, and the third reflected light individually. Alternatively, the photodetector may detect either one of the following types of light: light generated by superposition of the first reflected light, the second reflected light, and the third reflected light; and reflected light generated due to light generated by superposition of the first reflected light, the second reflected light, and the third reflected light. The expression “result of detection of the first reflected light, the second reflected light, and the third reflected light” may include a result represented by a signal generated in response to the photodetector detecting each of the first reflected light, the second reflected light, and the third reflected light. Alternatively, the expression may include a detection result represented by a signal generated in response to the photodetector detecting either one of the following types of light: reflected light generated due to light generated by superposition of the first reflected light, the second reflected light, and the third reflected light, and light generated by superposition of the first reflected light, the second reflected light, and the third reflected light. The third emitted light may have a wavelength that is the same as or different from the wavelength of each of the first emitted light and the second emitted light.

According to the configuration mentioned above, the addition of the third light source makes it possible to further increase the quantity of light, or acquire more information.

The first light emitter may further include a third light source that emits third emitted light, a fourth light source that emits fourth emitted light, a second sub-mount that supports the third light source and the fourth light source, and a housing that houses the first light source, the second light source, the third light source, and the fourth light source. The photodetector may further detect third reflected light, and fourth reflected light. The third reflected light emanates from the subject due to the third emitted light diffused by the diffuser. The fourth reflected light emanates from the subject due to the fourth emitted light diffused by the diffuser. The processing circuit may generate and output the information, based on a result of detection of the first reflected light, the second reflected light, the third reflected light, and the fourth reflected light by the photodetector. The first light source, the second light source, the third light source, and the fourth light source may be housed in the housing as a single package.

The photodetector may detect the first reflected light, the second reflected light, the third reflected light, and the fourth reflected light individually. Alternatively, the photodetector may detect either one of the following types of light: light generated by superposition of the first reflected light, the second reflected light, the third reflected light, and the fourth reflected light; and reflected light generated due to light generated by superposition of the first reflected light, the second reflected light, the third reflected light, and the fourth reflected light. The expression “result of detection of the first reflected light, the second reflected light, the third reflected light, and the fourth reflected light” may include a result represented by a signal generated in response to the photodetector detecting each of the first reflected light, the second reflected light, the third reflected light, and the fourth reflected light. Alternatively, the expression may include a detection result represented by a signal generated in response to the photodetector detecting either one of the following types of light: reflected light generated due to light generated by superposition of the first reflected light, the second reflected light, the third reflected light, and the fourth reflected light; and light generated by superposition of the first reflected light, the second reflected light, the third reflected light, and the fourth reflected light.

According to the configuration mentioned above, the addition of the fourth light source makes it possible to further increase the quantity of light, or acquire more information. Furthermore, the four light sources are integrated into a single package. This allows emitted light from each of the four light sources to be concentrated and incident on a narrow region of the diffuser.

The processing circuit may estimate a distance from the photodetector to at least one measurement point on the subject, based on the result of detection by the photodetector. The processing circuit may estimate a position of the at least one measurement point, based on the distance from the photodetector to the at least one measurement point on the subject that has been estimated by the processing circuit. The processing circuit may estimate a first incident angle and a second incident angle, based on the position estimated by the processing circuit. The first incident angle is an angle at which the first emitted light is incident on the at least one measurement point. The second incident angle is an angle at which the second emitted light is incident on the at least one measurement point. The processing circuit may correct a signal representative of the result of detection, based on the first incident angle estimated by the processing circuit, the second incident angle estimated by the processing circuit, and previously generated calibration data. The calibration data defines a relationship between an incident angle of light on the subject and a reflectance of the subject to the light. The processing circuit may generate the information, based on the signal corrected by the processing circuit.

The configuration mentioned above makes it possible to, based on the previously generated calibration data, suitably correct a signal output from the photodetector and, based on the corrected signal, generate more accurate information related to the subject.

The subject may be a living body, and the information may include, for example, at least one selected from the group consisting of blood flow, oxygen saturation, pulse rate, and blood pressure of the subject. The information may include information representative of brain blood flow of the living body.

The measurement apparatus may further include a second light emitter, and a mirror. The second light emitter includes a third light source that emits third emitted light, a fourth light source that is adjacent to the third light source and emits fourth emitted light, and a second sub-mount that supports the third light source and the fourth light source. The mirror causes the first emitted light, the second emitted light, the third emitted light, and the fourth emitted light to be concentrated and incident on one region of the diffuser, by changing a direction of propagation of at least one selected from the group consisting of light emitted from the first light emitter and light emitted from the second light emitter.

A measurement apparatus according to a still another aspect of the present disclosure includes a light source, a diffuser, a photodetector, and a processing circuit. The diffuser is disposed in an optical path of emitted light emitted from the light source. The photodetector detects reflected light emanating from the subject due to the emitted light diffused by the diffuser. The processing circuit generates and outputs information related to the subject, based on a result of detection of the reflected light by the photodetector. The processing circuit estimates a distance from the photodetector to at least one measurement point on the subject, based on the result of detection by the photodetector. The processing circuit estimates a position of the at least one measurement point, based on the distance estimated by the processing circuit. The processing circuit estimates an incident angle of the emitted light on the at least one measurement point, based on the position estimated by the processing circuit. The processing circuit corrects a signal representative of the result of detection, based on the incident angle estimated by the processing circuit, and previously generated calibration data. The calibration data defines a relationship between an incident angle of the emitted light on the subject and a reflectance of the subject to the emitted light. The processing circuit generates the information, based on the signal corrected by the processing circuit.

The configuration mentioned above makes it possible to, based on the previously generated calibration data, suitably correct a signal output from the photodetector and, based on the corrected signal, generate more accurate information related to the subject. According to the above-mentioned aspect, multiple light sources may not necessarily be provided. If multiple light sources are to be used, the above-mentioned signal correction may be performed for each light source, or for each wavelength to be used.

A method according to a still another aspect of the present disclosure includes executing a set of operations multiple times under multiple conditions, and generating calibration data. The set of operations includes: emitting light from a light source toward a subject or a plate to measure a reflectance for at least one measurement point on the subject or on the plate, the plate being used for calibration and similar in optical characteristics to the subject; and estimating an incident angle of the light on the at least one measurement point. The multiple conditions include varying a distance between the subject or the plate and the light source. The calibration data defines a relationship between the incident angle of the light on the at least one measurement point, and the reflectance.

The method mentioned above makes it possible to efficiently generate calibration data that is to be looked up when the measurement apparatus performs the signal correction mentioned above.

More specific embodiments of the present disclosure will be described below with reference to the drawings.

### EMBODIMENTS

### 1. Configuration of Measurement Apparatus

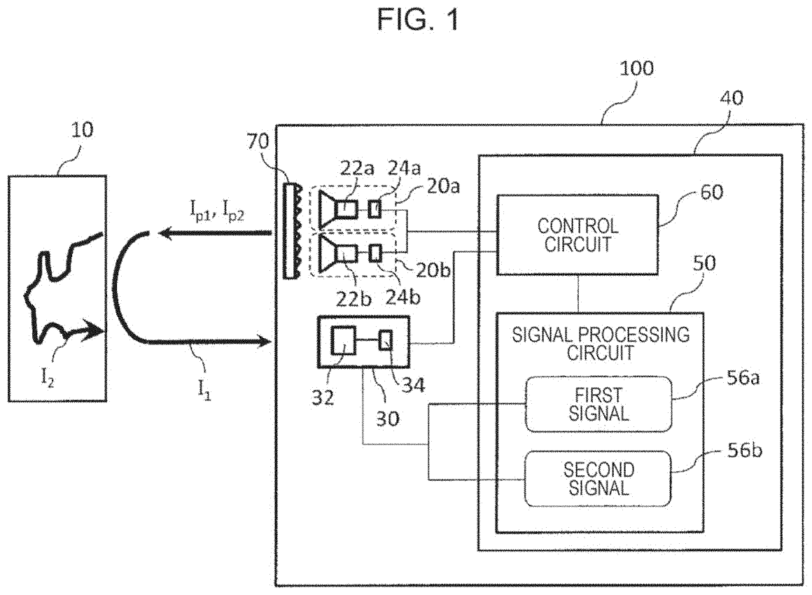

FIG. 1 schematically illustrates a measurement apparatus 100 according to an embodiment of the present disclosure. The measurement apparatus 100 according to the embodiment irradiates a target portion 10 of a subject of measurement with light, and detects light reflected from the target portion 10 to thereby acquire internal information on the target portion 10 in a non-contact manner. The subject of measurement may be, for example, a living body such as a human being. The target portion 10 may be, for example, the head of a human being, in particular, a part of the head where skin is exposed, such as the forehead. The internal information may, for example, reflect the condition of human brain activity. Depending on the intended application, the target portion 10 may be an object other than a living body. For example, a scatterer such as a liquid, a gas, or a food item may serve as the subject. Unless specified otherwise, the following description assumes that the target portion 10 is a human forehead.

The measurement apparatus 100 according to the embodiment includes a first light source 20a, a second light source 20b, a photodetector 30, an electronic circuit 40, and a diffuser 70. The electronic circuit 40 includes a signal processing circuit 50, and a control circuit 60. The first light source 20a and the second light source 20b will be hereinafter referred to, without distinction, as “light source 20” or “light sources 20a” in some cases. The first light source 20a and the second light source 20b each emit a light pulse for irradiating the target portion 10. The photodetector 30 detects part of a reflected light pulse reflected by the target portion 10, and outputs a signal representative of its intensity. The signal processing circuit 50 processes the signal output from the photodetector 30 to generate and output a signal representative of the internal condition of the target portion 10. The control circuit 60 controls the first light source 20a, the second light source 20b, the photodetector 30, and the signal processing circuit 50. Although not illustrated in FIG. 1, the measurement apparatus 100 may include, between the light source 20 and the diffuser 70, an optical system including one or more mirrors, one or more collimator lenses, or other optical elements. A specific configuration of the optical system will be described later.

Reference is now made to various components of the measurement apparatus 100. 1-1. First Light Source 20a and Second Light Source 20b

The first light source 20a emits a first light pulse Iₚ₁ for irradiating the target portion 10. The first light pulse Iₚ₁ has a first wavelength. The second light source 20b emits a second light pulse Iₚ₂ for irradiating the target portion 10. The second light pulse Iₚ₂ has a second wavelength greater than the first wavelength. Although FIG. 1 depicts an example in which a single first light source 20a is provided, multiple first light sources 20a may be provided. The same applies to the number of second light sources 20b. Using multiple light sources that emit light with the same wavelength makes it possible to increase the intensity of the light to be radiated. Depending on the intended application, rather than both the first light source 20a and the second light source 20b, only one of these two types of light sources may be used. In that case, two or more first light sources 20a, or two or more second light sources 20b are placed.

The first light pulse Iₚ₁ and the second light pulse Iₚ₂ will be hereinafter referred to, without distinction, as “light pulse Iₚ” or “light pulses Iₚ” in some cases. The light pulse Iₚ includes a rising part and a falling part. The rising part refers to a part of the light pulse Iₚ from the beginning of an increase in its intensity to the end of the increase. The falling part refers to a part of the light pulse Iₚ from the beginning of a decrease in its intensity to the end of the decrease.

The first light source 20a includes a first light-emitting element 22a, and a first regulator circuit 24a. The first light-emitting element 22a emits light with an intensity that varies with a first current injected into the first light-emitting element 22a. The first regulator circuit 24a regulates the first current. The second light source 20b includes a second light-emitting element 22b, and a second regulator circuit 24b. The second light-emitting element 22b emits light with an intensity that varies with a second current injected into the second light-emitting element 22b. The second regulator circuit 24b regulates the second current. The first light-emitting element 22a and the second light-emitting element 22b will be hereinafter referred to, without distinction, as “light-emitting element 22” or “light-emitting elements 22” in some cases. The first regulator circuit 24a and the second regulator circuit 24b will be hereinafter referred to, without distinction, as “regulator circuit” or “regulator circuits” in some cases. The first light-emitting element 22a and second light-emitting element 22b may each include, for example, a laser diode that emits laser light. The first regulator circuit 24a and the second regulator circuit 24b may each include, for example, a field effect transistor (FET) including a gallium nitride (GaN) semiconductor with superior electrical response.

When the light pulse Iₚ arrives at the target portion 10, a part of the light pulse Iₚ becomes a surface reflection component I₁, which is a component reflected at the surface of the target portion 10, and another part of the light pulse Iₚ becomes an internal scattering component I₂, which is a component reflected or scattered once inside the target portion 10 or scattered multiple times inside the target portion 10. The surface reflection component I₁ includes the three following sub-components: a regular reflection component, a diffuse reflection component, and a scattered reflection component. The regular reflection component refers to a reflection component whose angle of reflection is equal to the angle of incidence. The diffuse reflection component refers to a component that is diffused and reflected by irregularities present on the surface. The scattered reflection component refers to a component that is scattered and reflected by the internal tissues near the surface. If the target portion 10 is the user's forehead, the scattered reflection component refers to a component that is scattered and reflected inside the epidermis. The following description assumes that the surface reflection component I₁, which reflects off the surface of the target portion 10, includes these three sub-components. The following description also assumes that the internal scattering component I₂ does not include a component that is scattered and reflected by the internal tissues near the surface. Each of the surface reflection component I₁ and the internal scattering component I₂ changes its direction of travel when reflected or scattered, and a part of the component arrives at the photodetector 30 as a reflected light pulse. The surface reflection component I₁ may contain surface information on the subject, for example, face or scalp blood flow information. The internal scattering component I₂ may contain internal information on the subject, for example, brain blood flow information. Accordingly, detecting the surface reflection component I₁ makes it possible to acquire surface information on the subject, for example, face or scalp blood flow information. Detecting the internal scattering component I₂ makes it possible to acquire internal information on the subject, for example, brain blood flow information.

Each of the first wavelength of the first light pulse Iₚ₁, and the second wavelength of the second light pulse Iₚ₂ may be any wavelength within a wavelength range of greater than or equal to 650 nm and less than or equal to 950 nm. This wavelength range falls within the red to near-infrared wavelength range. The wavelength range mentioned above is called “biological window”, where there is relatively less absorption by water within a living body and by skin. For detection with a living body as a subject, use of the above-mentioned wavelength range allows for increased detection sensitivity. It is presumed that in detecting a change in user's brain blood flow, light used for the detection is mainly absorbed by oxygenated hemoglobin (HbO₂) and deoxygenated hemoglobin (Hb). A change in blood flow generally causes a change in the concentration of the oxygenated hemoglobin and a change in the concentration of deoxygenated hemoglobin. Such changes also cause a change in the degree of light absorption. Therefore, a change in blood flow also causes the quantity of detected light to change with time.

Oxygenated hemoglobin and deoxygenated hemoglobin differ in the wavelength dependence of light absorption. At wavelengths greater than or equal to 650 nm and less than 805 nm, deoxygenated hemoglobin has a coefficient of light absorption that is greater than the coefficient of light absorption by oxygenated hemoglobin. At the wavelength of 805 nm, deoxygenated hemoglobin and oxygenated hemoglobin have substantially the same coefficient of light absorption. At wavelengths greater than 805 nm and less than or equal to 950 nm, oxygenated hemoglobin has a coefficient of light absorption that is greater than the coefficient of light absorption by deoxygenated hemoglobin.

Accordingly, the first wavelength of the first light pulse Iₚ₁ may be set to a value greater than or equal to 650 nm and less than 805 nm, and the second wavelength of the second light pulse Iₚ₂ may be set to a value greater than or equal to 805 nm and less than or equal to 950 nm. By irradiating the target portion 10 with the first light pulse Iₚ₁ and the second light pulse Iₚ₂ each having the wavelength described above, the respective concentrations of oxygenated hemoglobin and deoxygenated hemoglobin that are contained in the blood inside the target portion 10 can be determined through a process that will be described later. The irradiation with two light pulses of different wavelengths makes it possible to acquire more detailed internal information on the target portion 10.

According to the embodiment, the first light source 20a and the second light source 20b may be designed by taking their potential effects on the user's retina into account. For example, the first light source 20a and the second light source 20b may be designed to satisfy the requirements for Class 1 of the laser safety standard developed in each individual country. If the requirements for Class 1 are satisfied, the target portion 10 is irradiated with light with such a low illuminance that the Accessible Emission Limit (AEL) is below 1 mW. The first light source 20a and the second light source 20b themselves may not necessarily satisfy the requirements for Class 1. For example, the diffuser 70 or a ND filter may be installed in front of the first light source 20a and the second light source 20b to diffuse or attenuate light so that the requirements for Class 1 of the laser safety standard are satisfied.

### 1-2. Photodetector 30

The photodetector 30 outputs a first signal 56a corresponding to the intensity of at least part of the components of a first reflected light pulse, which is generated by reflection of the first light pulse Iₚ₁ by the target portion 10. The photodetector 30 outputs a second signal 56b corresponding to the intensity of at least part of the components of a second reflected light pulse, which is generated by reflection of the second light pulse Iₚ₂ by the target portion 10. If the photodetector 30 detects at least part of the components of each of the first and second reflected light pulses in the falling period of the reflected light pulse, information on the blood flow inside the target portion 10 can be acquired. If the photodetector 30 detects at least part of the components of each of the first and second reflected light pulses in the rising period of the reflected light pulse, information on the blood flow at the surface of the target portion 10 can be acquired. The above-mentioned detecting operation of the photodetector 30 is controlled by the control circuit 60. The falling period of the first reflected light pulse or the second reflected light pulse is the period from when a decrease in the intensity of the first reflected light pulse or the second reflected light pulse begins to when the decrease ends. The rising period of the first reflected light pulse or the second reflected light pulse is the period from when an increase in the intensity of the first reflected light pulse or the second reflected light pulse begins to when the increase ends.

The measurement apparatus 100 may include two photodetectors 30. Of the two photodetectors 30, one may detect at least part of the components of the first reflected light in the falling period of the first reflected light, and outputs the detection result to the first signal 56a, and the other may detect at least part of the components of the second reflected light in the falling period of the second reflected light, and output the detection result to the second signal 56b. That is, according to the embodiment, the number of photodetectors 30 may not necessarily be one but may be two or more.

The photodetector 30 may include photoelectric converters 32, and charge storage sections 34. Specifically, the photodetector 30 may include a two-dimensional array of photodetection cells. Such configuration allows the photodetector 30 to detect two-dimensional information on the target portion 10 at once. A photodetection cell will be herein also referred to as “pixel”. The photodetector 30 may be, for example, any imager such as a CCD image sensor or a CMOS image sensor. In a more general arrangement, the photodetector 30 includes at least one photoelectric converter 32, and at least one charge storage section 34.

The photodetector 30 may include an electronic shutter. The electronic shutter is a circuit that controls the timing of image capture. The electronic shutter controls a period of single charge storage in which received light is converted into an effective electrical signal and stored, and a period in which signal storage is stopped. The signal storage period will be also referred to as “exposure period”. In the following description, the duration of the exposure period will be also referred to as “shutter duration”. The period of time from when one exposure period ends to when the next exposure period begins will be also referred to as “non-exposure period”.

The photodetector 30 allows the exposure period and the non-exposure period to be adjusted by means of the electronic shutter in a sub-nanosecond range, for example, in the range of 30 ps to 1 ns. Time-of-flight (TOF) cameras, which are used for distance measurement, detect all of light emitted from a light source and reflected back from a subject. Thus, for such a TOF camera, the shutter duration needs to be greater than the pulse duration of light. In contrast, the measurement apparatus 100 according to the embodiment does not require the shutter duration to be greater than the pulse duration. The shutter duration can be set to a value of, for example, greater than or equal to 1 ns and less than or equal to 30 ns. The measurement apparatus 100 according to the embodiment makes it possible to reduce the shutter duration, and consequently reduce the influence of dark current included in a detection signal.

The photodetector 30 may include a two-dimensional array of pixels on its imaging surface. Each pixel may include, for example, a photoelectric converter such as a photodiode, and one or more charge storage sections.

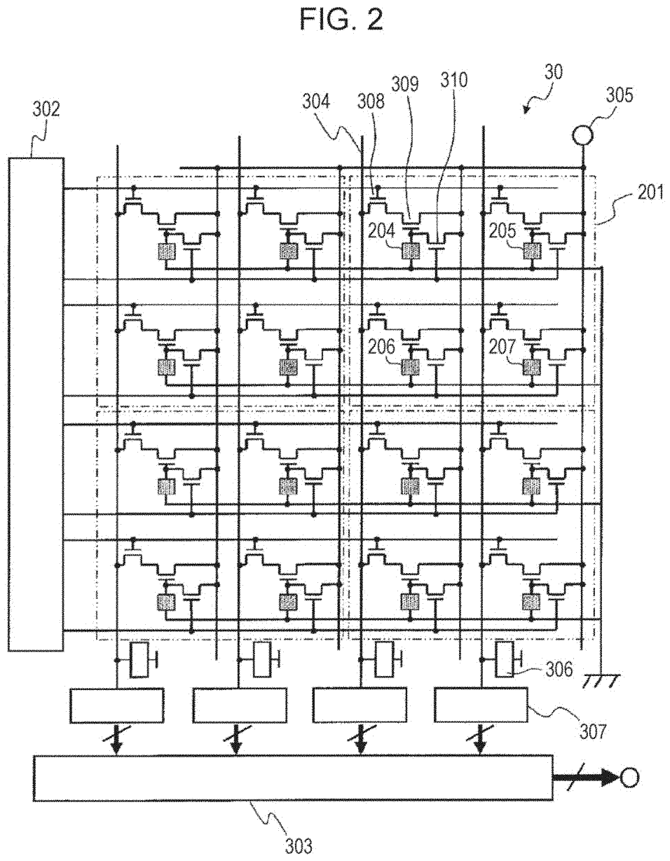

FIG. 2 illustrates an exemplary configuration of the photodetector 30. In FIG. 2, regions bounded by two-dot chain lines each correspond to a single pixel 201. The pixel 201 includes a single photodiode (not illustrated). Although FIG. 2 depicts only four pixels arranged in two rows and two columns, more pixels may be arranged in practice. The pixel 201 includes a first floating diffusion layer 204, a second floating diffusion layer 205, a third floating diffusion layer 206, and a fourth floating diffusion layer 207. Signals stored in the first to fourth floating diffusion layers 204 to 207 are treated as if the signals of four pixels of a typical CMOS image sensor, and are output from the photodetector 30. Although FIG. 2 illustrates an example in which each pixel 201 includes four floating diffusion layers, depending on the intended application, the pixel 201 may include three or more floating diffusion layers, or five or more floating diffusion layers. The number of floating diffusion layers to be included in each pixel 201 depends on the number of signals to be stored simultaneously.

Each pixel 201 includes four signal detection circuits. Each signal detection circuit includes a source follower transistor 309, a row selection transistor 308, and a reset transistor 310. A non-limiting example of each of these transistors may be a field-effect transistor formed on a semiconductor substrate. As illustrated in FIG. 2, one of the input terminal and the output terminal of the source follower transistor 309, and one of the input terminal and the output terminal of the row selection transistor 308 are connected with each other. The one of the input terminal and the output terminal of the source follower transistor 309 is typically a source. The one of the input terminal and the output terminal of the row selection transistor 308 is typically a drain. The gate of the source follower transistor 309, which serves as a control terminal, is connected with a photodiode. The signal charge of holes or electrons generated by the photodiode is stored in a floating diffusion layer, which is a charge storage section located between the photodiode and the source follower transistor 309.

Although not illustrated in FIG. 2, the first to fourth floating diffusion layers 204 to 207 are connected with a photodiode. A switch may be disposed between the photodiode and each of the first to fourth floating diffusion layers 204 to 207. The switch is designed to, in accordance with a charge storage pulse from the control circuit 60, switch the states of conduction between the photodiode and each of the first to fourth floating diffusion layers 204 to 207. This controls when to start and stop the storage of signal charge for each of the first to fourth floating diffusion layers 204 to 207. The electronic shutter according to the embodiment includes a mechanism for such exposure control.

The signal charge stored in each of the first to fourth floating diffusion layers 204 to 207 is read out as the gate of the row selection transistor 308 is turned on by a row selection circuit 302. At this time, the current flowing from a source follower power supply 305 into each of the source follower transistor 309 and a source follower load 306 is amplified in accordance with the signal potential of each of the first to fourth floating diffusion layers 204 to 207. An analog signal due to the current is read out from each vertical signal line 304, and converted into digital signal data by an analog-to-digital (AD) conversion circuit 307 connected for each individual column. The digital signal data is read out column by column by a column selection circuit 303 for output from the photodetector 30. The row selection circuit 302 and the column selection circuit 303 each first read out one column, and then read out the next column. Thereafter, the row selection circuit 302 and the column selection circuit 303 likewise read out information on the signal charge in each of the floating diffusion layers in all rows. After all signal charges are read out, the control circuit 60 turns on the gate of each reset transistor 310 to thereby reset all floating diffusion layers. This completes imaging of a single frame. Thereafter, high-speed frame imaging is repeated similarly to complete imaging of a series of frames by the photodetector 30.

Although the foregoing description of the embodiment is directed to an example of the photodetector 30 that is a CMOS photodetector, the photodetector 30 to be used may be another type of imager. The photodetector 30 may be, for example, any one of the following devices: a CCD photodetector; a single-photon counting device; and an amplifying image sensor such as an EMCCD or ICCD. If a single-pixel sensor is used as the photodetector 30, although there is only a single measurement point, high-speed detection is possible.

### 1-3. Electronic Circuit 40

The signal processing circuit 50 included in the electronic circuit 40 processes a signal output from the photodetector 30. The signal processing circuit 50 generates and outputs a signal representing the internal condition of the target portion 10, based on the first signal 56a and the second signal 56b output from the photodetector 30. It is assumed herein that the first light pulse Iₚ₁ has a wavelength of greater than or equal to 650 nm and less than 805 nm, and that the second light pulse Iₚ₂ has a wavelength greater than or equal to 850 nm and less than or equal to 950 nm. In this case, solving a predetermined system of equations by using the first signal 56a and the second signal 56b makes it possible to determine how much the respective concentrations of HbO₂ and Hb in blood have changed from their initial values. The system of equations is given by, for example, Equations (1) and (2) below.

$${ε}_{OXY}^{750} Δ Hb {O}_{2} + {ε}_{deOXY}^{750} Δ Hb = - ln \frac{{I}_{now}^{750}}{{I}_{ini}^{750}} \qquad ( 1 )$$

$${ε}_{OXY}^{850} Δ Hb {O}_{2} + {ε}_{deOXY}^{850} Δ Hb = - ln \frac{{I}_{now}^{850}}{{I}_{ini}^{850}} \qquad ( 2 )$$

ΔHbO₂ and ΔHb respectively represent how much the concentrations of HbO₂ and Hb in blood have changed from their initial values. ε⁷⁵⁰_{OXY} and ε⁷⁵⁰_{deOXY} respectively represent the molar absorption coefficients of HbO₂ and Hb at the wavelength of 750 nm. ε⁸⁵⁰_{OXY} and ε⁸⁵⁰_{OXY} respectively represent the molar absorption coefficients of HbO₂ and Hb at the wavelength of 850 nm. I⁷⁵⁰ᵢₙᵢ and I⁷⁵⁰_{now} respectively represent the detected intensities at the initial instant and at the instant of measurement with respect to the wavelength of 750 nm. I⁸⁵⁰ᵢₙᵢ and I⁸⁵⁰_{now} respectively represent the detected intensities at the initial instant and at the instant of measurement with respect to the wavelength of 850 nm. The present example assumes that the brain is not in an activated state at the initial instant and in an activated state at the instant of detection.

The signal processing circuit 50 may be implemented by, for example, a digital signal processor (DSP), a programmable logic device (PLD) such as a field programmable gate array (FPGA), or a combination of a central processing unit (CPU) or a graphics processing unit (GPU) and a computer program.

The control circuit 60 included in the electronic circuit 40 controls the first regulator circuit 24a and the second regulator circuit 24b so that the light pulse Iₚ is emitted from each of the first light-emitting element 22a and the second light-emitting element 22b. The control circuit 60 adjusts the time difference between the timing of emission of the light pulse Iₚ by each of the first light source 20a and the second light source 20b, and the shutter timing of the photodetector 30. The above-mentioned time difference will be herein sometimes referred to as “phase difference”. The “timing of emission” by the first light source 20a and the second light source 20b refers to the timing when the light pulse emitted from each of the first light source 20a and the second light source 20b starts to rise. The “shutter timing” refers to the timing to start exposure. The control circuit 60 may adjust the phase difference by varying the timing of emission, or may control the phase difference by varying the shutter timing.

The control circuit 60 may be designed to remove an offset component from a signal detected by each pixel of the photodetector 30. The offset component refers to a signal component due to ambient light, such as sunlight or fluorescent light, or due to disturbance light. The offset component due to ambient light or disturbance light is estimated by the photodetector 30 detecting a signal with the first light source 20a and the second light source 20b being turned off, that is, with no light being emitted from the first light source 20a and the second light source 20b.

The control circuit 60 may be, for example, a combination of a processor and a memory, or an integrated circuit such as a microcontroller incorporating a processor and a memory. In one example, as the processor executes a program stored in the memory, the control circuit 60 performs operations such as adjusting the timing of emission and the shutter timing, and causing the signal processing circuit 50 to execute signal processing.

The signal processing circuit 50 and the control circuit 60 may be a single unitary circuit, or may be separate discrete circuits. The signal processing circuit 50 may be, for example, a component of an external apparatus, such as a server disposed at a remote location. In this case, the external apparatus such as a server transmits and receives data to and from the light source 20, the photodetector 30, and the control circuit 60 via wireless or wired communications.

### 1-4. Other Components

The measurement apparatus 100 may include imaging optics for forming a two-dimensional image of the target portion 10 on the light-receiving surface of the photodetector 30. The optical axis of the imaging optics is substantially orthogonal to the light-receiving surface of the photodetector 30. The imaging optics may include a zoom lens. A change in the location of the zoom lens causes a change in the magnification of the two-dimensional image of the target portion 10, which in turn causes a change in the resolution of the two-dimensional image on the photodetector 30. This ensures that even if the target portion 10 is at a far distance, a desired measurement region can be magnified for detailed observation.

The measurement apparatus 100 may include a bandpass filter between the target portion 10 and the photodetector 30. The bandpass filter is designed to pass only light within a wavelength range emitted by the first light source 20a and the second light source 20b, or light with wavelengths in the vicinity of the wavelength range. This helps to reduce the influence of ambient light or other disturbance components. The bandpass filter may be, for example, a multilayer filter or an absorption filter. In consideration of a band shift due to temperature variation of each of the first light source 20a and the second light source 20b or oblique incidence on the filter, the bandpass filter may be designed to have a bandwidth of greater than or equal to about 20 nm and less than or equal to about 100 nm.

The measurement apparatus 100 may include a polarizer disposed between the target portion 10 and each of the first light source 20a and the second light source 20b, and a polarizer disposed between the target portion 10 and the photodetector 30. In this case, the polarization direction of the polarizer disposed near the first light source 20a and the second light source 20b, and the polarization direction of the polarizer disposed near the photodetector 30 may have a crossed-Nicols relationship. The positioning of these two polarizers helps to ensure that the regular reflection component contained in the surface reflection component I₁ reflected from the target portion 10, that is, a component with the same angle of reflection as the angle of incidence, does not arrive at the photodetector 30. In other words, the quantity of the surface reflection component I₁ of light that arrives at the photodetector 30 can be reduced.

### 2. Signal Detection Operation

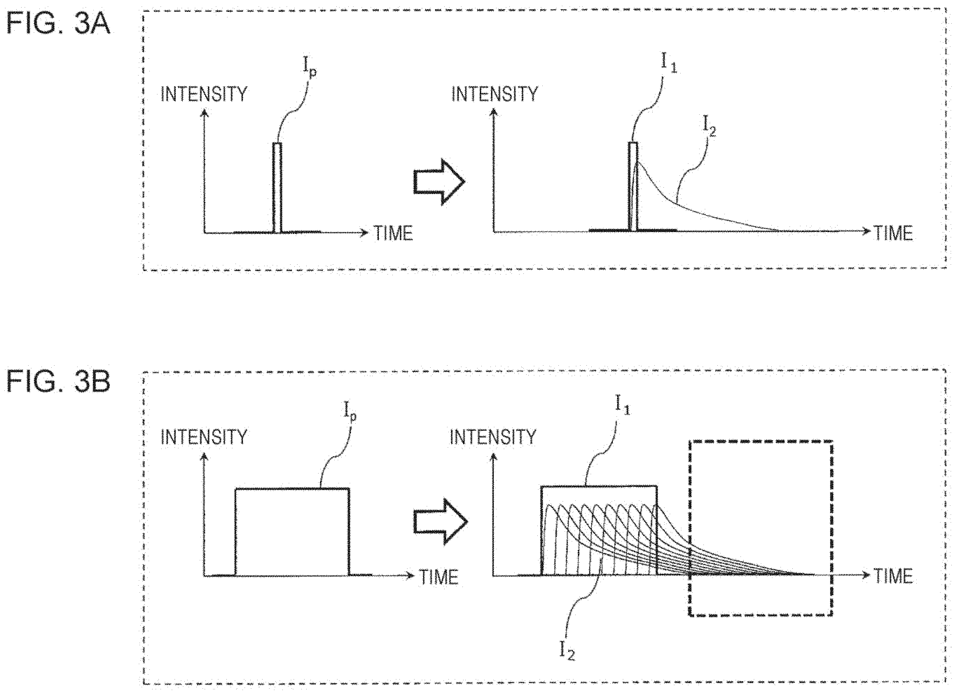

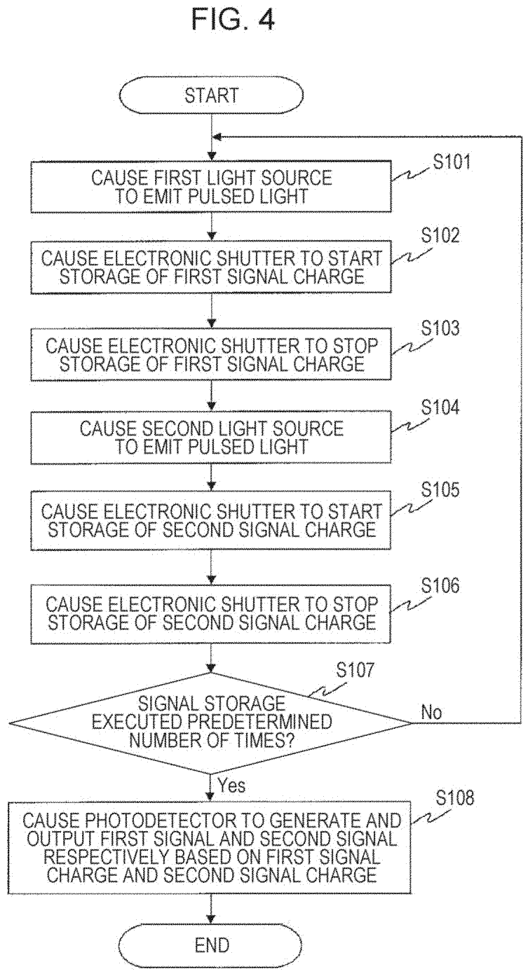

Reference is now made to FIGS. 3A to 4 to describe an exemplary operation of detecting a biometric signal. A specific exemplary method for detecting the internal scattering component I₂ is described below.

FIG. 3A schematically illustrates, for a case where the light pulse Iₚ has an impulse waveform, exemplary time variations of the surface reflection component I₁ and the internal scattering component I₂ that are contained in a reflected light pulse. FIG. 3B schematically illustrates, for a case where the light pulse Iₚ has a rectangular waveform, exemplary time variations of the surface reflection component I₁ and the internal scattering component I₂ that are contained in the reflected light pulse. In each of FIGS. 3A and 3B, the left-hand side of the figure represents an example of the waveform of the light pulse Iₚ emitted from each of the first light source 20a and the second light source 20b, and the right-hand side of the figure represents an example of the respective waveforms of the surface reflection component I₁ and the internal scattering component I₂ that are contained in the reflected light pulse. Although the internal scattering component I₂ has a very low intensity in practice, the intensity of the internal scattering component I₂ is depicted in exaggerated form in FIGS. 3A and 3B.

As illustrated in FIG. 3A, if the light pulse Iₚ has an impulse waveform, the surface reflection component I₁ is similar in waveform to the light pulse Iₚ, and the internal scattering component I₂ has an impulse response waveform with a delay relative to the surface reflection component I₁. This is because the internal scattering component I₂ corresponds to a combination of rays that have passed through various paths inside the target portion 10.

As illustrated in FIG. 3B, if the light pulse Iₚ has a rectangular waveform, the surface reflection component I₁ is similar in waveform to the light pulse Iₚ, and the internal scattering component I₂ has a waveform that is a superposition of multiple impulse response waveforms. The inventors have confirmed that such superposition of multiple impulse response waveforms makes it possible to amplify the quantity of the internal scattering component I₂ of light detected by the photodetector 30. Starting the electronic shutter at the rising part of the reflected light pulse allows for effective detection of the internal scattering component I₂. A region enclosed by dashed lines at the right-hand side of FIG. 3B represents an exemplary shutter opening period in which the electronic shutter of the photodetector 30 is opened. In comparison to using light sources such as femtosecond lasers with a pulse duration of the order of femtoseconds (fs) or picosecond lasers with a pulse duration of the order of picoseconds (ps), using light sources that emit rectangular pulses with a duration of the order of 1 ns to 10 ns as the first and second light sources 20a and 20b allows the first and second light sources 20a and 20b to be driven at a comparatively low voltage. This makes it possible to reduce the size and cost of the measurement apparatus 100.

Streak cameras are used in the art to distinguish between and detect pieces of information such as light absorption coefficients or light scattering coefficients at different locations along the depth inside a living body. For example, Japanese Unexamined Patent Application Publication No. 4-189349 discloses an example of such a streak camera. Streak cameras typically employ ultrashort pulses of light with a duration of the order of femtoseconds or picoseconds to enable measurement with a desired spatial resolution. In contrast, the embodiment makes it possible to distinguish between and detect the surface reflection component I₁ and the internal scattering component I₂. Therefore, the first light source 20a and the second light source 20b are not necessarily required to emit ultrashort pulses of light, and any suitable pulse duration may be chosen.

In irradiating a human head with light to acquire brain blood flow information, the quantity of the internal scattering component I₂ of light can have a very small value, which is about a few thousandths to a few tenths of thousandths of that of the surface reflection component I₁. With the laser safety standard further taken into account, the quantity of light that can be radiated is very small. This makes detection of the internal scattering component I₂ very difficult. Even for such cases, making each of the first light source 20a and the second light source 20b emit the light pulse Iₚ with a relatively long pulse duration makes it possible to increase the integrated quantity of the internal scattering component I₂ having a relative time delay. This helps to increase the quantity of detected light, and improve the SN ratio.

The first light source 20a and the second light source 20b are capable of, for example, emitting the light pulse Iₚ with a pulse duration of greater than or equal to 3 ns. Alternatively, the first light source 20a and the second light source 20b may emit the light pulse Iₚ with a pulse duration of greater than or equal to 5 ns, or with an even longer pulse duration of greater than or equal to 10 ns. An excessively long pulse duration, however, leads to waste as the quantity of unused light increases. Accordingly, the first light source 20a and the second light source 20b may be, for example, controlled to emit the light pulse Iₚ with a pulse duration of less than or equal to 50 ns. Alternatively, the first light source 20a and the second light source 20b may emit the light pulse Iₚ with a pulse duration of less than or equal to 30 ns, or with an even shorter pulse duration of less than or equal to 20 ns. If the rectangular pulse has a pulse duration of a few ns to a few tens of ns, the first light source 20a and the second light source 20b can be driven at a comparatively low voltage. This makes it possible to reduce the size and cost of the measurement apparatus 100.

The radiation pattern of each of the first light source 20a and the second light source 20b may be, for example, a pattern that provides a uniform intensity distribution within a region to be irradiated. The measurement apparatus 100 according to the embodiment differs in this respect from, for example, the conventional apparatus disclosed in Japanese Unexamined Patent Application Publication No. 11-164826. With the apparatus disclosed in Japanese Unexamined Patent Application Publication No. 11-164826, the detector and the light source are about 3 cm apart, and the surface reflection component is spatially separated from the internal scattering component. This necessarily results in discrete radiation of light. In contrast, the embodiment allows the surface reflection component I₁ to be temporally separated from the internal scattering component I₂ and reduced. This in turn allows use of light sources with a radiation pattern that provides a uniform intensity distribution. Such a radiation pattern with a uniform intensity distribution may be produced by the diffuser 70 diffusing light emitted from each of the first light source 20a and the second light source 20b.

Unlike with techniques according to the related art, the embodiment makes it possible to detect the internal scattering component I₂ even directly under the irradiation point on the target portion 10. The embodiment also makes it possible to enhance measurement resolution by irradiating the target portion 10 with light over a spatially large area.

FIG. 4 is a flowchart illustrating an overview of how the control circuit 60 operates in controlling the first light source 20a, the second light source 20b, and the photodetector 30. The control circuit 60 executes the operations schematically illustrated in FIG. 4 to cause the photodetector 30 to detect at least part of the components of the first and second reflected light pulses in the respective falling periods of the first and second reflected light pulses.

At step S101, the control circuit 60 causes the first light source 20a to emit the first light pulse Iₚ₁ for a predetermined time. At this time, the photodetector 30 is in a state with exposure stopped by the electronic shutter. The control circuit 60 causes the electronic shutter to stop exposure until the completion of a period of time taken for the surface reflection component I₁ of the first pulsed light to arrive at the photodetector 30. Next, at step S102, the control circuit 60 causes the electronic shutter to start exposure at the timing when the internal scattering component I₂ of the first reflected light pulse arrives at the photodetector 30. The control circuit 60 thus causes the electronic shutter to start storage of a signal charge. The signal charge stored at this time will be hereinafter referred to as “first signal charge”. After elapse of a predetermined time, at step S103, the control circuit 60 causes the electronic shutter to stop the exposure. This causes the electronic shutter to stop the storage of the first signal charge. Through steps S102 and S103, the first signal charge is stored into one of the first to fourth floating diffusion layers 204 to 207 illustrated in FIG. 2.

At step S104, the control circuit 60 causes the second light source 20b to emit the second light pulse Iₚ₂ for a predetermined time. At this time, the photodetector 30 is in a state with exposure stopped by the electronic shutter. The control circuit 60 causes the electronic shutter to stop exposure until the completion of a period of time taken for the surface reflection component I₁ of the second pulsed light to arrive at the photodetector 30. Next, at step S105, the control circuit 60 causes the electronic shutter to start exposure at the timing when the internal scattering component I₂ of the second reflected light pulse arrives at the photodetector 30. The control circuit 60 thus causes the electronic shutter to start storage of a signal charge. The signal charge stored at this time will be hereinafter referred to as “second signal charge”. After elapse of a predetermined time, at step S106, the control circuit 60 causes the electronic shutter to stop the exposure. This causes the electronic shutter to stop the storage of the second signal charge. Through steps S105 and S106, the second signal charge is stored into another one of the first to fourth floating diffusion layers 204 to 207 illustrated in FIG. 2.

Subsequently, at step S107, the control circuit 60 determines whether the above-mentioned signal storage has been executed a predetermined number of times. If the result of the determination at step S107 is No, steps S101 to S106 are repeated until the result of the determination becomes Yes.

If the result of determination at step S107 is Yes, the process proceeds to step S108. At step S108, the control circuit 60 causes the photodetector 30 to generate and output the first signal 56a and the second signal 56b based on the first signal charge and the second signal charge, respectively.

As described above, the control circuit 60 executes a first operation of causing the first light source 20a to emit the first light pulse Iₚ₁, and causing the photodetector 30 to detect at least part of the components of the first reflected light pulse in the falling period of the first reflected light pulse. The control circuit 60 executes a second operation of causing the second light source 20b to emit the second light pulse Iₚ₂, and causing the photodetector 30 to detect at least part of the components of the second reflected light pulse in the falling period of the second reflected light pulse. The control circuit 60 repeats a series of operations including the first operation and the second operation a predetermined number of times. Alternatively, the control circuit 60 may repeat the first operation a predetermined number of times, and then repeat the second operation a predetermined number of times. The first operation and the second operation may be interchanged in their order.

The operations illustrated in FIG. 4 allow the internal scattering component I₂ to be detected with high sensitivity. In irradiating a human head with light to acquire information such as brain blood flow, the light undergoes a very large attenuation inside the human head. For example, the light exiting the head may, in some cases, be attenuated in intensity to about one millionth of the light entering the head. Thus, in some cases, radiation of a single pulse alone may not provide a sufficient quantity of light for detecting the internal scattering component I₂. For radiation according to Class 1 of the laser safety standard, in particular, the quantity of light provided is very small. Accordingly, in the example illustrated in FIG. 4, the first light source 20a and the second light source 20b each emit a light pulse multiple times, and in a similar manner, the photodetector 30 is also exposed multiple times by means of the electronic shutter. Through such operations, the detection signal can be integrated for improved sensitivity. Executing emission of light and exposure multiple times is not necessarily required but may be performed as needed.

Although FIG. 4 illustrates an example in which the internal scattering component 12 is detected, in another example, the surface reflection component I₁ may be detected. In another example, both the surface reflection component I₁ and the internal scattering component I₂ may be detected. If both components are to be detected, a step of storing a signal charge based on the surface reflection component I₁ of the first reflected light pulse is added between step S101 and step S102, and a step of storing a signal charge based on the surface reflection component I₁ of the second reflected light pulse is added between step S104 and step S105. These signal charges are stored into the two remaining floating diffusion layers of the first to fourth floating diffusion layers 204 to 207 illustrated in FIG. 2. Detecting the surface reflection component I₁ makes it possible to, for example, acquire information representing the outward appearance of a face or the state of scalp blood flow.

Although the example described above is directed to use of two light sources that emit light with different wavelengths, alternatively, multiple light sources that emit light with the same wavelength may be used. If the quantity of light provided by each individual light source is small, using multiple light sources that emit light with the same wavelength helps to compensate for the shortage of the quantity of light. The term “same wavelength” as used herein does not necessarily mean strictly the same wavelength but may mean slightly different wavelengths.

3. Concentration of Light onto Diffuser 70

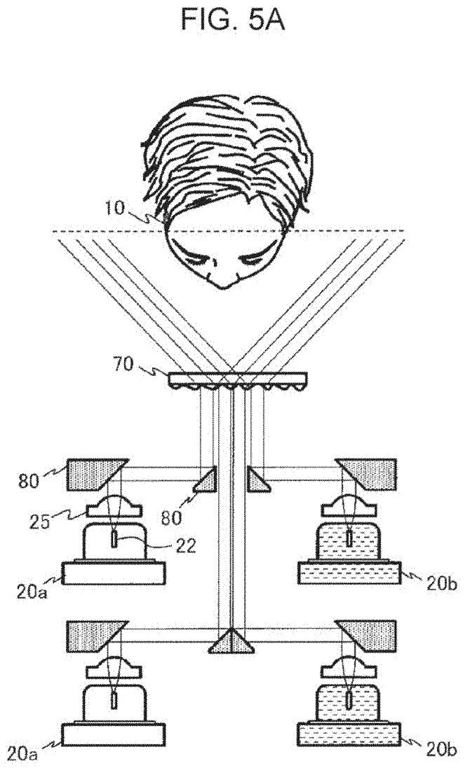

Reference is now made to FIGS. 5A and 5B to describe an exemplary configuration for concentrating light onto a diffuser 70.

FIG. 5A illustrates an exemplary configuration of a diffuser 70, light sources, and an optical system. A measurement apparatus 100 according to the present example includes two first light sources 20a that each emit light with a first wavelength, and two second light sources 20b that each emit light with a second wavelength. The first wavelength is, for example, a wavelength within the wavelength range from 650 nm to 805 nm, and the second wavelength is, for example, a wavelength within the wavelength range from 805 nm to 950 nm. The optical system includes mirrors 80, and collimator lenses 25. The mirrors 80 direct first emitted light emitted from the first light sources 20a and second emitted light emitted from the second light sources 20b, such that the first emitted light and the second emitted light are concentrated and incident on one region of the diffuser 70. The collimator lenses 25 each collimate light emitted from the corresponding first light source 20a or the corresponding second light source 20b into parallel light.

To perform non-contact measurement at a distance as with the embodiment, a high-power light source is required to ensure that the light to be diffused have sufficient intensity. Accordingly, in the example illustrated in FIG. 5A, the first light sources 20a that emit light with the same first wavelength, and the second light sources 20b that emit light with the same second wavelength are provided.

The collimator lenses 25 are disposed in one-to-one correspondence to the light sources 20. Each collimator lens 25 collimates light emitted from the corresponding first light source 20a or the corresponding second light source 20b. The mirrors 80 are each disposed in the optical path between the corresponding first light sources 20a or the corresponding second light source 20b and the diffuser 70. The mirrors 80 each reflect a bundle of rays collimated by the corresponding collimator lens 25 to thereby change the direction of propagation of the bundle of rays. The presence of the collimator lenses 25 and the mirrors 80 allows bundles of rays from the first light source 20a and the second light source 20b to be concentrated onto the diffuser 70. This makes it possible to ensure that the distance between the center of a light spot formed on the diffuser 70 by the first emitted light, and the center of a light spot formed on the diffuser 70 by the second emitted light is less than the distance between the center of the corresponding first light source 20a and the center of the corresponding second light source 20b.

Due to constraints on light source package size, it is typically not possible to make the spacing between bundles of rays from light sources less than the spacing between the light sources. In contrast, according to the embodiment, the presence of the optical system including the collimator lenses 25 and the mirrors 80 allows the spacing between bundles of rays from light sources to be less than the spacing between the light sources. For example, the spacing between the bundles of rays on the diffuser 70 can be made less than or equal to the spacing between the light sources. Concentrating the bundles of rays onto one region on the diffuser 70 by the optical system as described above allows light to shine from the region toward the target portion 10.

According to the embodiment, bundles of rays from the first light sources 20a and the second light sources 20b are made to emanate from substantially the same point on the diffuser 70. This means that for any light source, light from the light source is incident on the target portion 10 at substantially the same incident angle. The difference in incident angle between the light sources is thus reduced. This helps to ensure that even if the target portion 10 and the measurement apparatus 100 change in their relative positions due to body motion or motion of the measurement apparatus 100, variation of illuminance or reflectance on the target portion 10 does not depend on the difference in light quantity or illuminance non-uniformity between the light sources. This facilitates correction, that is, calibration of a signal acquired by the photodetector 30. This effect is particularly pronounced for the target portion 10 with a curved shape. The illuminance on the target portion 10 increases as the incident angle of light approaches the perpendicular. If, unlike with the example illustrated in FIG. 5A, the target portion 10 is irradiated with rays of light from two distant points on the diffuser 70, one of the rays of light from the two points that impinges on the target portion 10 at an angle closer to the perpendicular has the greater influence on the illuminance on the target portion 10. Accordingly, if the target portion 10 and the two points change in their relative positions due to body motion or motion of the measurement apparatus 100, this causes a change in the difference between the angles at which the rays of light from the two points are incident on the target portion 10. This results in variation of the respective contributions of the light components from the two distant points on the diffuser 70, which in turn causes the illuminance on the target portion 10 to change in a complex manner. In contrast, according to the embodiment, bundles of rays from the first light sources 20a and the second light sources 20b are concentrated onto one region of the diffuser 70 as illustrated in FIG. 5A. This helps to reduce the influence of the difference in intensity between the light sources on the variation of illuminance on the target portion 10 associated with body motion or motion of the measurement apparatus 100.

The diffuser 70 has depressions or projections on its surface to diffuse an incident ray in diverse random directions. The diffuser 70 thus serves to eliminate or reduce illuminance non-uniformity on the target portion 10. The diffuser 70 may be in the form of, for example, an array of microlenses. As with the example illustrated in FIG. 5A, the diffuser used may be of a type that refracts light therein and transmits the refracted light. Alternatively, other than such a type of diffuser, a diffuser of a type that diffuses light by reflection may be used. Such a reflective diffuser also has minute irregularities on its reflecting surface so that the reflected light is diffused in random directions.

Although it would be possible to use a lens instead of the diffuser 70 to diffuse light, a lens typically causes the illuminance distribution of light from a light source to be projected directly without being changed. Illuminance non-uniformity thus tends to remain on the target portion 10. Consequently, if multiple light sources are used and one of the light sources decreases in intensity, the two-dimensional distribution of the illuminance on the target portion 10 itself may also change, which may cause deviation from the calibration data. In contrast, according to the embodiment, the diffuser 70 is used as described above. In this case, although variation of overall intensity associated with a change in the intensity of one or more of multiple light sources still occurs, as for the two-dimensional distribution of illuminance, a distribution according to the characteristics of the diffuser 70 can be obtained in a stable manner. This therefore helps to reduce deviation of the two-dimensional distribution from the calibration data.

The optical system according to the embodiment causes bundles of rays from the first light sources 20a and the second light sources 20b to impinge on the diffuser 70 in a substantially parallel-directed manner. If the bundles of rays do not impinge on the diffuser 70 in a parallel-directed manner, light emerging from the diffuser 70 is diffused but tends to exhibit an illuminance distribution that is dependent on the angle of incidence on the diffuser 70. That is, an illuminance distribution that is dependent on the angle of incidence on the diffuser 70 tends to remain. According to the embodiment, the collimator lenses 25 are used to not only concentrate bundles of rays but also cause bundles of rays to impinge on the diffuser 70 in a parallel-directed manner. This configuration evenly reduces the influence of non-uniformity of the distribution of illuminance provided by each light source. This configuration also helps to ensure that, even if there is a difference in the intensity of emitted light between light sources, the influence of such difference in intensity between the light sources on the distribution of illuminance on the surface of the target portion 10 is not likely to manifest itself in comparison to a case where bundles of rays do not impinge on the diffuser 70 in a parallel-directed manner. That is, rays of light from individual light sources are diffused from substantially the same region with substantially the same distribution in a superposed manner. In particular, if the relative intensity difference between the light sources varies with time, non-uniformity of the angle of incidence on the diffuser 70 tends to cause corresponding variation of the two-dimensional distribution of illuminance on the surface of the target portion 10. This results in deviation from previously retained calibration data used for calibration of the illuminance distribution. In this regard, if incident light is collimated, this helps to reduce time variation of the relative two-dimensional distribution of illuminance after emergence of light from the diffuser 70. Errors in the correction of the illuminance distribution can be thus reduced.

Each collimator lens 25 according to the embodiment is disposed immediately after the light-emitting element 22 of each of the corresponding first light sources 20a and the corresponding second light source 20b. Emitted light is thus collimated before being reflected by the corresponding mirror 80. This configuration helps to reduce the width of the bundle of rays from each light source. This allows the bundles of rays from the light sources to be concentrated at a higher density.

The reflecting surface of each mirror 80 may have any shape such as a square or a rectangle. If high-power semiconductor lasers are used as the first light sources 20a and the second light sources 20b, the radiation of rays from each light-emitting element 22 tends to spread out in a direction perpendicular to a surface of the sub-mount in contact with the light-emitting element 22. A collimated bundle of the rays thus has an elliptical cross-sectional profile. Accordingly, the length and width of the reflecting surface of each mirror 80 may be adjusted in accordance with the elliptical profile. In comparison to using mirrors having a square reflecting surface, using the mirrors 80 having a rectangular reflecting surface with its length and width adjusted appropriately makes it possible to place the mirrors 80 in closer proximity to each other, and consequently concentrate the bundle of rays onto the diffuser 70 at a higher density. Instead of using the mirrors 80 with a planar reflecting surface, mirrors with a free-form surface may be used. By forming the reflecting surface of each mirror 80 as a curved surface adapted to the angle of radiation of light from the corresponding light source, it is possible to collimate the reflected rays. This allows the collimator lenses 25 to be eliminated. By adjusting the curvature radius of the curved surface to match each of the vertical and horizontal focal lengths for rays of light emitted from the light sources, it is possible to collimate the rays with greater precision.

Use of an optical fiber is another conceivable method for concentrating light emitted from the first light sources 20a and the second light sources 20b onto one location of the diffuser 70. The use of an optical fiber to concentrate light, however, may result in a significantly reduced degree of parallelism of rays of light incident on the diffuser 70. For this reason, the embodiment achieves concentration of light not by use of an optical fiber but by use of an optical system including the collimator lenses 25 and the mirrors 80.

The number of first light sources 20a and the number of second light sources 20b may not necessarily be two but may be one, or may be greater than or equal to three. Increasing the number of first light sources 20a and the number of second light sources 20b makes it possible to increase the intensity of emitted light. Only either the first light sources 20a or the second light sources 20b may be provided. In that case, multiple light sources that emit light with the same wavelength are provided.

According to the embodiment, light beams emitted from the first light sources 20a, and light beams emitted from the second light sources 20b are concentrated onto a relatively narrow region of the diffuser 70 by the corresponding collimator lenses 25 and the corresponding mirrors 80. This facilitates the consistency of the illuminance distribution on the target portion 10 between the two wavelengths. FIG. 5B illustrates exemplary illuminance distributions for a location represented by dashed lines in FIG. 5A. In FIG. 5B, reference sign I₂₀ₐ represents an exemplary illuminance distribution for light emitted from the first light sources 20a, and reference sign I_{20b} represents an exemplary illuminance distribution for light emitted from the second light sources 20b. According to the embodiment, two wavelengths of light are made to emanate from substantially the same point on the diffuser 70. This means that even if the target portion 10 and the measurement apparatus 100 change in their relative positions, the illuminance ratio between the two wavelengths is always constant on the target portion 10. For example, even when rays of light are incident on a side of the target portion 10, the angle of incidence of the rays on the side is substantially equal between the two wavelengths. Consequently, in comparison to a case where bundles of rays of two wavelengths are incident at distant locations on the diffuser 70, the above-mentioned configuration facilitates the consistency of the reflectance between the two wavelengths. According to the embodiment, when body motion or motion of the measurement apparatus 100 occurs, detection signals corresponding to the two wavelengths change in synchronism. This helps to reduce signal noise associated with such body motion or motion of the measurement apparatus 100.

Reference is now made to FIG. 5C to describe, in more detail, light concentrated onto the diffuser 70.

FIG. 5C schematically illustrates an example of two adjacent light spots 151 and 152 out of four light spots formed on one region of the diffuser 70. Each of the light spots 151 and 152 is formed as the corresponding one of two light beams emitted from two adjacent ones of multiple light sources is projected onto the diffuser 70. The light spots 151 and 152 have a distance “d” between each other that is less than the distance between the two adjacent light sources. For example, the distance “d” may be less than or equal to 5 mm. Two adjacent light spots will be herein regarded as being located at substantially the same position if the distance “d” between the light spots is less than or equal to 5 mm.

The greater the angle of inclination of the target portion 10, the greater the sensitivity of a change in illuminance or reflectance on the target portion 10 associated with body motion. In other words, in performing measurement with higher precision, the steeper the angle of inclination of the target portion 10, the smaller the tolerance allowed for the difference between the incident angle at which light from each first light source 20a is incident on the target portion 10, and the incident angle at which light from each second light source 20b is incident on the target portion 10. The difference in incident angle between these two light sources depends on the distance “d” between the light spots 151 and 152 on the diffuser. Accordingly, it is desirable to set the distance “d” to an appropriate value.

FIG. 5D is a graph representing, with a human forehead as a target portion, the simulated results on the relationship between the angle of inclination of the human forehead, and a tolerance M for the distance “d” between two light spots on the diffuser that correspond to different wavelengths. The tolerance M represents such a value of the distance “d” between the light spots that, before and after a human makes a 10-degree rotational motion in the horizontal direction, the difference between the rate of change of a detection signal due to the first emitted light, and the rate of change of a detection signal due to the second emitted light can be kept within 0.5%. In other words, if the distance “d” between the two light spots is less than the tolerance M, the difference in rate of change between these detection values can be kept to less than or equal to 0.5%. Consequently, an error in signal detection due to the difference in rate of change between these detection values can be made less than the amount of variation of the detection signal of brain blood flow. This allows a brain blood flow signal to be detected with greater precision. The graph in FIG. 5D represents the simulated results for the tolerance M calculated under the assumption that the detection signal attenuates progressively with cos θ with dependence on the incident angle θ at which emitted light is incident on the target portion.

The average forehead inclination angle of Caucasians based on MRI structure images is approximately 37 degrees near Area 46 for working memory in the frontal lobe. It is appreciated from FIG. 5D that when the forehead inclination angle is 37 degrees, the tolerance M is 5 mm. Therefore, making the distance “d” between the light spots less than or equal to 5 mm helps to ensure that even if a human generates a body motion, before and after the body motion, an error resulting from the difference in the rate of change in detection signal between two light sources does not exceed the amount of variation of the detection signal of brain blood flow. This enables brain blood flow measurement with high precision.

The distance “d” between these light spots may be less than or equal to 2 mm. The light spots 151 and 152 typically have an elliptical shape. The light spots 151 and 152 respectively have widths of w1 and w2 along their major axes. In the illustrated example, the distance “d” between the two light spots 151 and 152 is less than the widths w1 and w2. The two light spots 151 and 152 have an overlapping portion 153. As with the present example, the optical system can be designed such that rays of light emitted from multiple light sources are incident on the diffuser 70 in partially overlapping relation to each other. This allows diffuse light due to multiple emergent light beams to shine from substantially the same point on the diffuser 70.

The first light source 20a, the second light source 20b, the optical system, and the diffuser 70 may not necessarily be configured as described above but may be modified in various ways. Other exemplary configurations are now described below.

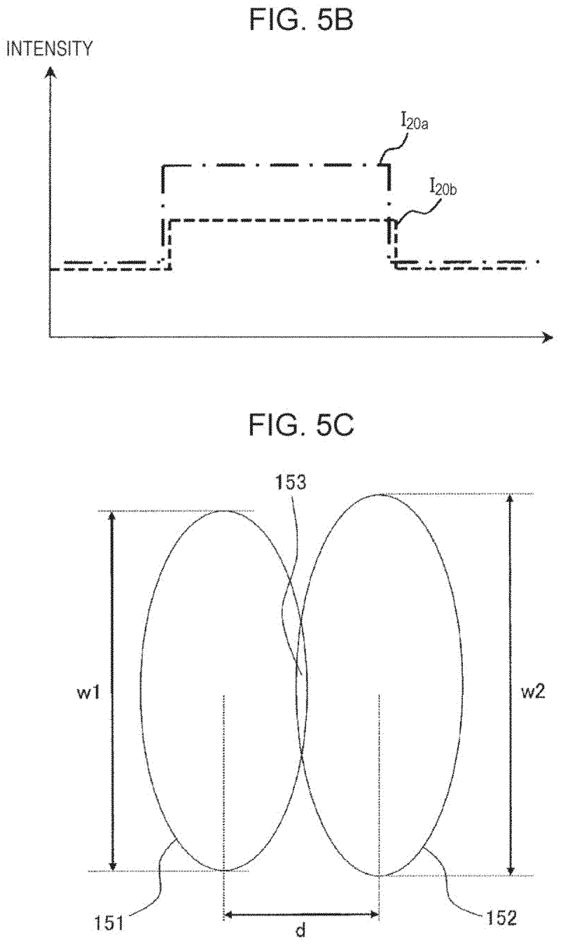

FIG. 6 illustrates another exemplary configuration of a diffuser 70, light sources 20, and an optical system. The optical system according to the example includes collimator lenses 25, mirrors 80 serving as first mirrors, and a second mirror 81. According to the example, the light sources 20 are arranged in one direction. The mirrors 80 are disposed in one-to-one correspondence to the light sources 20. The second mirror 81 further reflects a bundle of rays reflected by each first mirror 80 so that the bundle of rays is incident on the diffuser 70. Each light source 20 is at a different distance from the corresponding mirror 80. This helps to avoid interference between the paths of rays from the light sources 20, and achieve concentration of groups of rays from the light sources 20 to thereby enhance illuminance. Since the second mirror 81 reflects multiple bundles of rays at once, the number of components in the optical system can be reduced, leading to reduced cost.

The embodiment mentioned above employs the optical system including the mirrors 80 that causes light from the light sources 20 to be concentrated and incident on the diffuser 70. Alternatively, concentration of light may be accomplished without use of such an optical system. For example, a light emitter with multiple light sources integrated into a single package may be used. Use of such a light emitter also allows light emitted from multiple light sources to be concentrated and incident on one location of the diffuser 70.

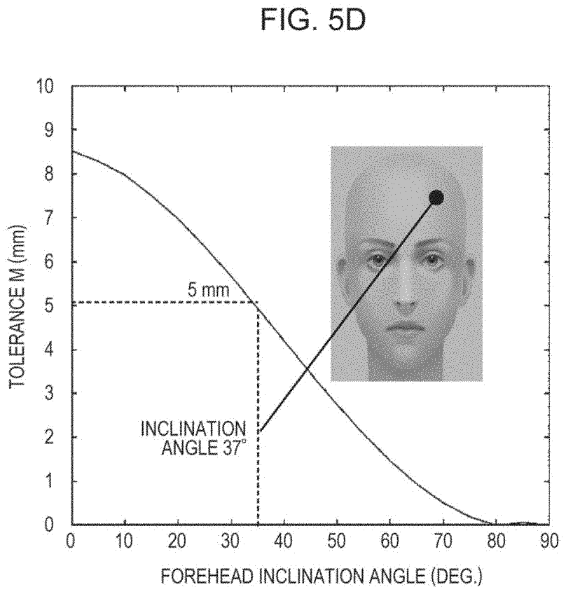

FIG. 7 illustrates an exemplary configuration of a light emitter 120 with multiple light-emitting elements 22 integrated into a single package. In the present example, the light-emitting elements 22 are arranged in one direction in proximity to each other. Each light-emitting element 22 may be, for example, a semiconductor laser. The light-emitting elements 22 are disposed on the same sub-mount, and integrated into a single package. No mirror for changing the path of light emitted from each light-emitting element 22 is disposed. This configuration allows emission points inside the package to be positioned in proximity to each other. According to this configuration as well, the collimator lenses 25 are each disposed between the diffuser 70 and the corresponding one of the light-emitting elements 22 to allow collimated light to be incident on the diffuser 70. The collimator lenses 25 are disposed in one-to-one correspondence to the light-emitting elements 22. This allows rays of light emitted from the light-emitting elements 22 to be collimated to reduce the cross-sectional area of a bundle of such rays.

The sub-mount is, for example, a component for supporting the first light-emitting element 22a and the second light-emitting element 22b. Since the first light-emitting element 22a and the second light-emitting element 22b are supported by the same component, these light-emitting elements can be positioned in proximity to each other. This allows the respective optical axes of the first light source 20a and the second light source 20b to be positioned close to each other to thereby reduce the distance between the light spot 151 formed by the first emitted light and the light spot 152 formed by the second emitted light. As a result, the first emitted light and the second emitted light are made to emanate from substantially the same point on the diffuser 70. This helps to reduce the difference in incident angle between rays of light incident on the target portion 10.

The sub-mount may be a component with heat-dissipating property. Using, for example, a semiconductor laser as each light-emitting element 22 may lead to thermally induced deterioration of light emission characteristics. The use of a component with heat-dissipating property as the sub-mount helps to reduce such thermally induced deterioration of the light emission characteristics of the semiconductor laser. The sub-mount may be made of a material with superior thermal conductivity, examples of which include ceramic materials such as aluminum nitride (AlN) and aluminum oxide (Al₂O₃), Cu—AlN—Cu (copper-aluminum nitride-copper) multilayer materials with layers of copper stacked on aluminum nitride, and composite metallic materials such as copper-tungsten (Cu—W) and copper-diamond (Cu-Diamond). This arrangement allows for efficient release of heat generated by the first light source 20a and the second light source 20b. The sub-mount is not limited to the above examples but may be in the form of a planar plate, a heat sink, or a surface-mount substrate.

The first light-emitting element 22a and the second light-emitting element 22b may be formed on a single laser chip or on a single semiconductor crystal. This allows the two light-emitting elements to be positioned in closer proximity to each other, which helps to more significantly reduce the difference in incident angle between rays of light incident on the target portion 10.

Presence of too many emission points may lead to deterioration of the characteristics of the rising part of the pulse waveform if there is a time difference between pulses of light emitted from the light-emitting elements 22. Accordingly, the output power per light-emitting element 22 may be maximized to minimize the number of light-emitting elements 22. For example, the number of light-emitting elements 22 within a single light source package may be less than or equal to ten, or may be less than or equal to five.

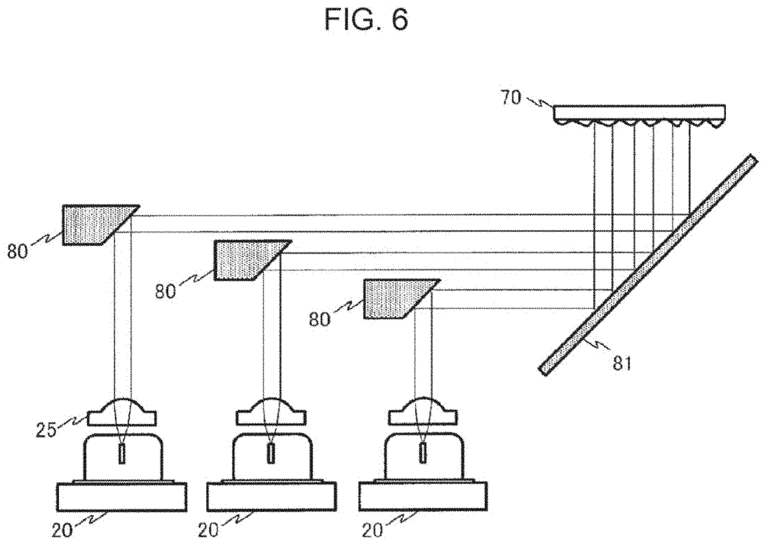

FIG. 8 illustrates another exemplary configuration of light emitters 120. A measurement apparatus 100 according to the present example includes four packages of the light emitters 120 mounted on a substrate 26. The four light emitters 120 are arranged in matrix form. Two light emitters 120 are arranged in each of the X-direction and the Y-direction illustrated in FIG. 8. Each light emitter 120 may be, for example, a semiconductor laser package. Each light emitter 120 includes a first light-emitting element 22a, a second light-emitting element 22b, a sub-mount 23 for supporting the first light-emitting element 22a and the second light-emitting element 22b, and a housing 27 for accommodating the first and second light-emitting elements 22a and 22b and the sub-mount 23. The first light-emitting element 22a emits light with a first wavelength. The second light-emitting element 22b emits light with a second wavelength. In each of the X-direction and the Y-direction, two adjacent light emitters 120 are mounted on the substrate 26 in 180-degree inverted positions relative to each other. Such positioning compensates for the difference between adjacent light emitters 120 in the illuminance distribution of light emitted from the first light-emitting element 22a and the second light-emitting element 22b. This facilitates the consistency of illuminance distribution on the target portion 10 between the two wavelengths. The number of light emitters 120 may not necessarily be four. Any number of light emitters 120 may be used.

Each of the first light-emitting elements 22a is electrically connected with a first regulator circuit 24a used to regulate a first current, and emits light of an intensity corresponding to the first current that has been injected. Likewise, each of the second light-emitting elements 22b is electrically connected with a second regulator circuit 24b used to regulate a second current, and emits light of an intensity corresponding to the second current that has been injected. The presence of the first regulator circuit 24a and the second regulator circuit 24b allows the first light-emitting element 22a and the second light-emitting element 22b on the same sub-mount to be driven appropriately based on what is to be measured. More specifically, injection of the first current from the first regulator circuit 24a, and injection of the second current from the second regulator circuit 24b are performed alternately so that light can be alternately emitted from the first light-emitting element 22a and the second light-emitting element 22b that are disposed on the same sub-mount. A problem associated with measurement using simultaneously emitted rays of light with different wavelengths is that the rays of light with different wavelengths are detected by the photodetector 30 as their sum, which makes it difficult to distinguish between the measurement results corresponding to individual wavelengths. Accordingly, the above-mentioned control is performed by using the first regulator circuit and the second regulator circuit to allow the first emitted light and the second emitted light to be emitted alternately. This advantageously helps to ensure that the measurement results corresponding to the light components of two wavelengths can be acquired individually through time division without being mixed together.

As described above, the first light-emitting element 22a and the second light-emitting element 22b on the same sub-mount are respectively controlled by means of the first regulator circuit 24a and the second regulator circuit 24b. This configuration makes it possible to more significantly reduce the difference in incident angle between rays of light incident on the target portion 10. Further, this configuration allows the measurement results corresponding to the light components of two wavelengths to be acquired individually without being mixed together.

### 4. Signal Calibration

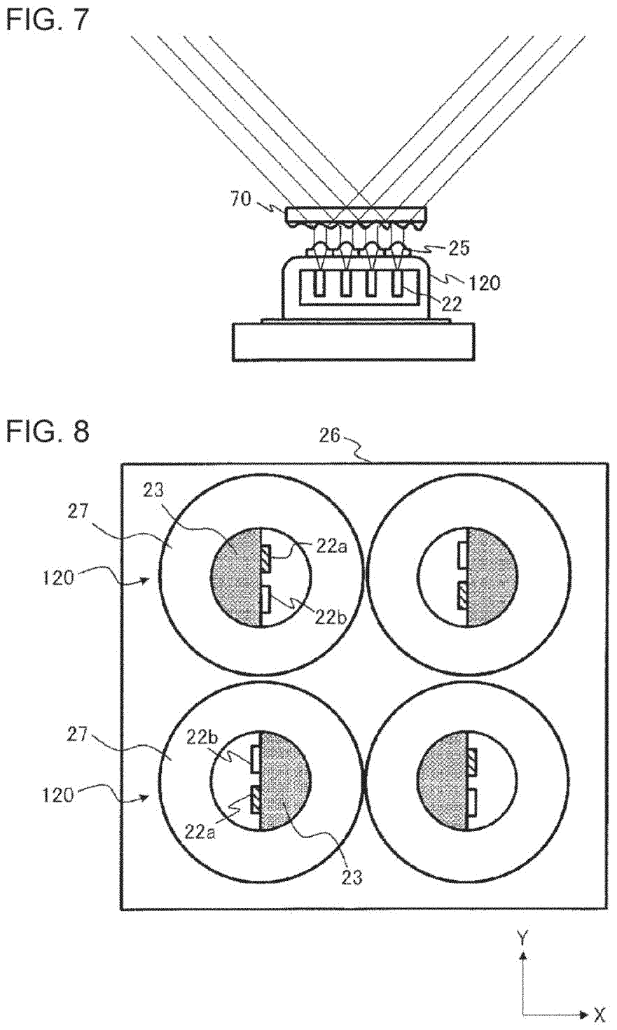

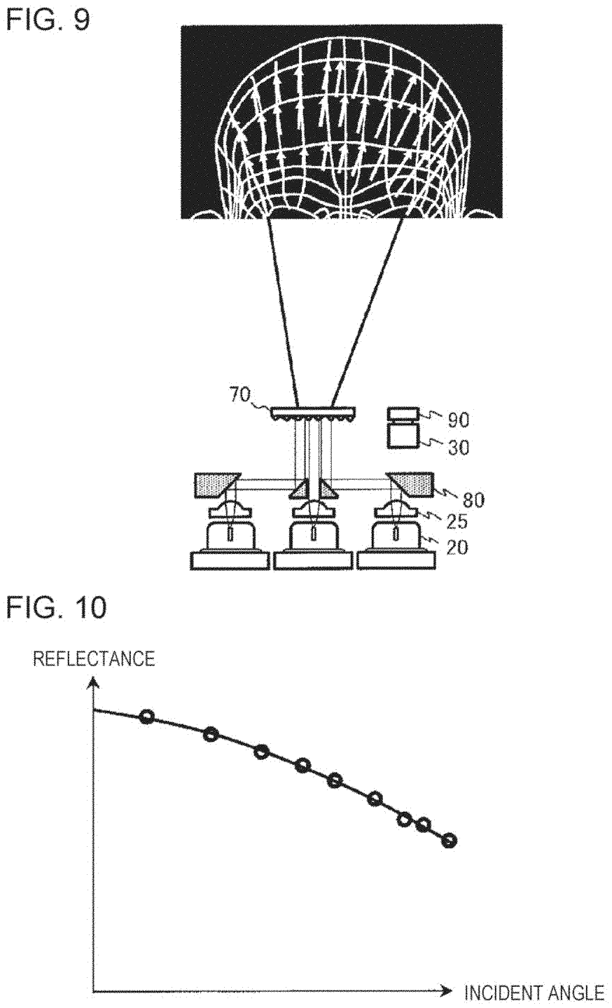

Reference is now made to FIGS. 9 and 10 to describe an exemplary calibration method for a signal detected by the photodetector 30.

According to the embodiment, calibration data is generated prior to measurement. The calibration data defines the relationship between the incident angle of light on the target portion 10, and reflectance. In measurement, the signal processing circuit 50 looks up the previously generated calibration data to correct a signal acquired from the photodetector 30. This allows a stable biometric signal to be acquired even when body motion or motion of the measurement apparatus 100 occurs. To execute signal calibration, the measurement apparatus 100 includes the capability to perform TOF ranging. The ranging is, for example, executed by the signal processing circuit 50 based on the result of detection of light emitted from either one of the first light source 20a and the second light source 20b. The TOF method used may be indirect TOF, or may be direct TOF.

FIG. 9 schematically illustrates how rays of light diffused by the diffuser 70 are incident on the target portion 10. Arrows in FIG. 9 represent the distribution of the surface normal vectors for the target portion 10. In the example illustrated in FIG. 9, three light sources 20 are arranged in one direction, and rays of light emitted from the three light sources 20 are concentrated by the mirrors 80 for incidence on the diffuser 70. The photodetector 30, and a lens 90 are disposed near the diffuser 70. The lens 90 focuses light from the target portion 10 to form an image on the light-receiving surface of the photodetector 30.

FIG. 10 is a graph illustrating the incident-angle dependence of the diffuse reflectance of the target portion 10 according to the configuration illustrated in FIG. 9. According to the embodiment, calibration data representing the incident-angle characteristics of diffuse reflectance as illustrated in FIG. 10 is generated in advance, and stored into a storage medium. In measurement, a signal is corrected by using the previously stored calibration data. The diffuse reflectance has a value that varies with the surface roughness of the target portion 10. Accordingly, data representing the incident-angle dependence of the diffuse reflectance may be acquired for each individual user as calibration data.

The calibration data according to the embodiment may be in the form of a table or a function that defines the relationship between the incident angle at which a bundle of rays is incident on the target portion 10, and the diffuse reflectance corresponding to the incident angle. The incident angle can be determined by use of, for example, TOF ranging. The measurement apparatus 100 is capable of determining the distance distribution of the target portion 10 through TOF imaging by use of pulses of light, and transforming the distance distribution into the distribution of three-dimensional coordinates. From the distribution of three-dimensional coordinates, the surface normal vector for each individual measurement point can be calculated. From the calculated surface normal vector, the three-dimensional coordinates of each individual point on the diffuser 70 from which a ray emanates, and the three-dimensional coordinates of each individual measurement point on the target portion 10, the incident angle at which a bundle of rays is incident on each individual measurement point can be determined. In the measurement apparatus 100 according to the embodiment, rays of light from the light sources 20 are concentrated onto one location of the diffuser 70, and light is incident on the target portion 10 from the location. Consequently, the incident angle can be uniquely determined for each individual measurement point on the target portion 10. Therefore, it is possible to acquire data representing the incident-angle dependence of diffuse reflectance from a single image of the target portion 10. This allows the calibration data to be acquired in a short time with little burden on the user. The table or function representing the incident-angle dependence of diffuse reflectance can be obtained by plotting the relationship between the incident angle and the signal value detected at each individual measurement point, and interpolating values between plotted points.

TOF ranging and signal detection can be executed by the same measurement apparatus 100. This helps to mitigate pixel misalignment, parallax, and occlusion to enable accurate calculation of the incident angle.

In the example illustrated in FIG. 9, the measurement apparatus 100 includes the lens 90 positioned to face the photodetector 30. The lens 90 forms, onto the light-receiving surface of the photodetector 30, an image of light reflected from the target portion 10. The photodetector 30 is thus able to acquire information representing the two-dimensional distribution of the intensity of light reflected from the target portion 10.

As with the example illustrated in FIG. 9, the lens 90 may be positioned in proximity to the diffuser 70. Such positioning helps to reduce the difference between the angle at which each ray impinges on the target portion 10 from the diffuser 70, and the angle at which each ray emanates from the target portion 10 toward the lens 90. This allows for stable acquisition of the data of the table representing the incident-angle dependence of diffuse reflectance.

The distance between the center of a group of rays concentrated onto the diffuser 70, and the center of the lens 90 may be less than or equal to 30 mm in one example, less than or equal to 20 mm in another example, or less than or equal to 10 mm in still another example.

The measurement apparatus 100 may emit light in the form of a continuous wave (CW) rather than a pulse to acquire information on the target portion 10. Use of light in a CW form allows the measurement apparatus 100 to be used also in applications for acquiring other vital information such as oxygen saturation, pulse rate, or blood pressure. In that case as well, concentrating multiple bundles of rays on the diffuser 70 is effective in achieving stable measurement with body motion components removed.

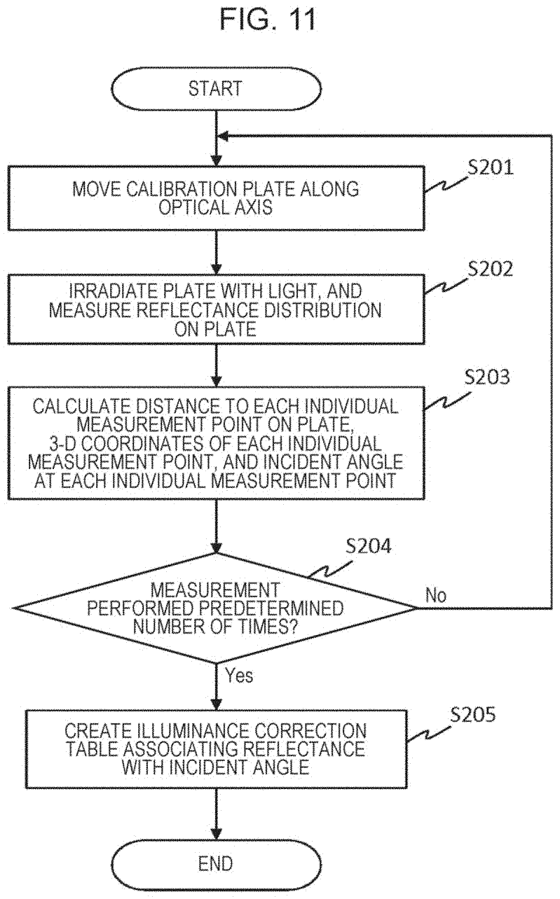

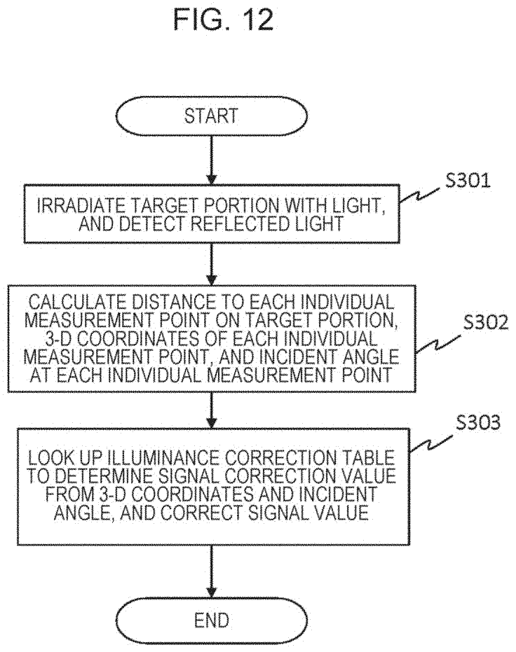

Reference is now made to FIGS. 11 and 12 to describe an exemplary method for generating calibration data, and an exemplary method for correcting a signal by use of the calibration data.

FIG. 11 is a flowchart illustrating an exemplary method for generating calibration data. In this example, a calibration plate, which is a plate used for calibration, is prepared in advance. The calibration plate has optical characteristics similar to the optical characteristics of the target portion 10 of a subject of measurement. An illuminance correction table is created as the calibration data. The illuminance correction table associates the illuminance or reflectance at each measurement point on the target portion 10 with the incident angle at the measurement point.

First, at step S201, the calibration plate is moved along the optical axis, and positioned at a predetermined location. This movement may be performed either automatically or manually. At the next step S202, the calibration plate is irradiated with light from the light source 20 of the measurement apparatus 100, and the reflectance distribution on the plate is measured with the photodetector 30. Then, at step S203, the signal processing circuit 50 calculates the distance from the photodetector 30 to each individual measurement point on the calibration plate, the three-dimensional coordinates of each individual measurement point, and the incident angle at each individual measurement point. The three-dimensional coordinates of each individual measurement point may be calculated based on the distance to the measurement point measured by TOF ranging, and the position of the measurement point within an image. The incident angle is calculated from the three-dimensional coordinates of the point of interest, and the three-dimensional coordinates of a point on the diffuser 70 from which light emanates. At the next step S204, the control circuit 60 determines whether the measurement has been performed a predetermined number of times. If the measurement has not been performed a predetermined number of times, the process returns step S201, and the calibration plate is moved by a predetermined distance along the optical axis. Until it is determined at step S204 that the measurement has been performed a predetermined number of times, the operations from steps S201 to S204 are repeated. Once the measurement has been performed a predetermined number of times, the process proceeds to step S205. At step S205, the signal processing circuit 50 creates an illuminance correction table that associates the reflectance at each individual measurement point measured at step S202, with the incident angle at each individual measurement point calculated at step S203. The illuminance correction table may be generated under multiple conditions of different distances between the calibration plate and the measurement apparatus 100.

If the measurement apparatus 100 includes multiple light sources that emit light with difference wavelengths, the operations in FIG. 11 may be executed for each light source to create the illuminance correction table.

The method described above makes it possible to effectively generate calibration data used to correct a detection signal output from the photodetector 30 during measurement.

Instead of the calibration plate, the target portion 10 of the subject itself may be used to generate calibration data by the same method as mentioned above.

As described above, the method for generating calibration data according to the embodiment includes executing a set of operations multiple times under multiple conditions, and generating calibration data. The set of operations includes: emitting light from a light source toward a subject of measurement or toward a plate to measure the reflectance for at least one measurement point on the subject or on the plate, the plate being used for calibration and similar in optical characteristics to the subject; and estimating the incident angle of the light on the at least one measurement point. The multiple conditions include varying the distance between the subject or the plate and the light source. The calibration data defines the relationship between the incident angle of the light on the at least one measurement point, and the reflectance. The method mentioned above makes it possible to efficiently generate calibration data.

FIG. 12 is a flowchart illustrating an exemplary method for signal correction that is executed during measurement by use of calibration data. In this example, first, at step S301, the target portion 10 is irradiated with light from the light source 20, and the light reflected by the target portion 10 is detected by the photodetector 30. At the next step S302, the signal processing circuit 50 calculates the distance from the photodetector 30 to each individual measurement point on the target portion 10, the three-dimensional coordinates of each individual measurement point, and the incident angle at each individual measurement point. Then, at step S303, the signal processing circuit 50 looks up a previously generated illuminance correction table to determine a signal correction value from the three-dimensional coordinates and the incident angle, and corrects the value of a signal generated by the photodetector 30. The correction is made by, for example, multiplying the signal value by the inverse of the reflectance illustrated in FIG. 10. The signal processing circuit 50 generates information representative of the internal condition of the subject based on the signal that has been corrected.

As described above, the signal processing circuit 50 according to the embodiment estimates the distance to at least one measurement point on a subject, based on the result of detection by the photodetector 30. The signal processing circuit 50 estimates the position of the at least one measurement point, based on the estimated distance. The signal processing circuit 50 estimates the incident angle of emitted light on the at least one measurement point, based on the estimated position. The signal processing circuit 50 corrects a signal representative of the result of detection of reflected light, based on the estimated incident angle and previously generated calibration data, which defines a relationship between incident angle and reflectance. The signal processing circuit 50 generates information related to the subject, based on the corrected signal. The operations mentioned above allows for appropriate signal correction even if motion of the target portion 10 or motion of the measurement apparatus 100 occurs.

### Modifications

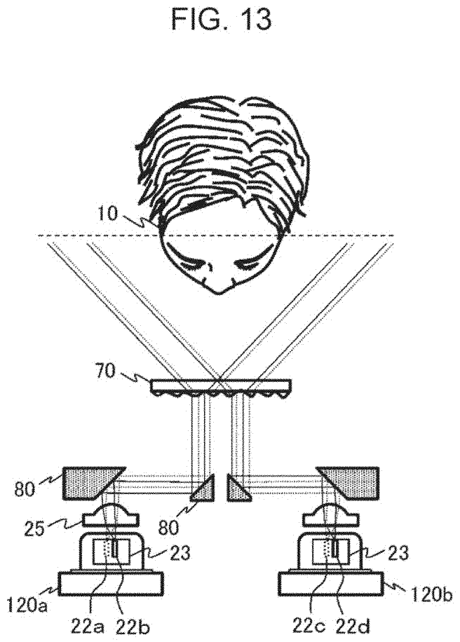

FIG. 13 illustrates another exemplary configuration of a measurement apparatus 100. The measurement apparatus 100 according to the present example includes a light emitter 120a, a light emitter 120b, a diffuser 70, mirrors 80, and collimator lenses 25. Each of the light emitter 120a and the light emitter 120b may be, for example, a semiconductor laser package. The light emitter 120a includes a first light-emitting element 22a, a second light-emitting element 22b, and a sub-mount 23 for supporting the first light-emitting element 22a and the second light-emitting element 22b. The light emitter 120b includes a third light-emitting element 22c, a fourth light-emitting element 22d, and a sub-mount 23 for supporting the third light-emitting element 22c and the fourth light-emitting element 22d. First emitted light emitted from the first light-emitting element 22a, and third emitted light emitted from the third light-emitting element 22c have, for example, a first wavelength. Second emitted light emitted from the second light-emitting element 22b, and fourth emitted light emitted from the fourth light-emitting element 22d have, for example, a second wavelength. The mirrors 80 cause the first emitted light and the second emitted light to be concentrated and incident on one region on the diffuser 70, and cause the third emitted light and the fourth emitted light to be concentrated and incident on one region on the diffuser 70. At this time, if any two light spots are selected from the group consisting of a light spot formed by the first emitted light, a light spot formed by the second emitted light, a light spot formed by the third emitted light, and a light spot formed by the fourth emitted light, the distance between the two selected light spots is less than the distance between light-emitting elements corresponding one-to-one to the selected light spots. For example, two light spots respectively formed on one region of the diffuser 70 by the first emitted light and the third emitted light have a distance between each other that is less than the distance between the first light-emitting element and the third light-emitting element. Two light spots respectively formed on one region of the diffuser 70 by the first emitted light and the fourth emitted light have a distance between each other that is less than the distance between the first light-emitting element and the fourth light-emitting element. Two light spots respectively formed on one region of the diffuser 70 by the second emitted light and the third emitted light have a distance between each other that is less than the distance between the second light-emitting element and the third light-emitting element. Two light spots respectively formed on one region of the diffuser 70 by the second emitted light and the fourth emitted light have a distance between each other that is less than the distance between the second light-emitting element and the fourth light-emitting element.

As described above, according to the present exemplary configuration, multiple light emitters 120 each including multiple light-emitting elements are incorporated into the measurement apparatus 100. This configuration makes it possible to increase the quantity of light radiated onto the target portion 10, and consequently improve the SN ratio of a signal observed by the photodetector 30. Further, the presence of mirrors allows light from the light emitter 120a and the light emitter 120b to be concentrated and incident on one region of the diffuser 70. This helps to ensure that, before and after body motion occurs, an error resulting from the difference in the rate of change in detection signal between the two light sources, that is, the light emitter 120a and the light emitter 120b, does not exceed the amount of variation of the detection signal of brain blood flow.

Multiple mirrors may not necessarily be provided. In one alternative example, concentration of emitted light onto one region of the diffuser 70 may be achieved by an arrangement in which no mirror is disposed in the optical path of the first emitted light and the second emitted light and a mirror is disposed in the optical path of the third emitted light and the fourth emitted light. The light emitter 120a and the light emitter 120b may be housed in a single housing.

The foregoing description of the embodiment is directed to an example of generating information related to blood flow of a subject, with the subject being mainly a living body. The technique according to the present disclosure is not limited in scope to biometric measurements but may find utility in measurement of the internal conditions of various substances. For example, the technique according to the present disclosure is applicable to measurement of the condition of fruits, vegetables, meat, fish, or cooked food. More specifically, the technique according to the present disclosure can be used to generate information such as the degree of spoilage in fresh food, processed food, or cooked food, or, for example, how thoroughly a food to be cooked (e.g., the dough of a cake sponge or frozen food) has been cooked or heated inside an oven. In another example, the subject may be the trunk, stems, or leaves of a plant, and information such as the internal growth of the plant can be generated. For the exemplary applications mentioned above, not only a single wavelength of light but also greater than or equal to two wavelengths of light may be used. If multiple wavelengths of light are to be used, wavelengths with different absorption coefficients are selected. With one of these wavelengths serving as a reference wavelength, internal condition can be estimated based on the relative amounts of light absorption at other wavelengths.

The measurement apparatus according to the present disclosure enables stable detection of information representing the internal condition of a subject even if motion of the subject or motion of a measurement apparatus occurs. The technique according to the present disclosure thus has utility in a wide variety of applications, including performing brain blood flow measurement, vital sensing, authentication, and food inspection in a non-contact manner.

What is claimed is:

1. A measurement apparatus comprising:
a first light source that emits first emitted light:
a second light source that emits second emitted light;
a diffuser;
a mirror that causes the first emitted light and the second emitted light to be concentrated and incident on one region of the diffuser, by changing a direction of propagation of at least one selected from the group consisting of the first emitted light and the second emitted light;
a photodetector that detects first reflected light and second reflected light, the first reflected light emanating from a subject due to the first emitted light diffused by the diffuser, the second reflected light emanating from the subject due to the second emitted light diffused by the diffuser; and
a processing circuit that, based on a result of detection of the first reflected light and the second reflected light by the photodetector, generates and outputs information related to the subject,
wherein the first emitted light forms a first light spot on the one region of the diffuser,
wherein the second emitted light forms a second light spot on the one region of the diffuser, and
wherein a distance between the first light spot and the second light spot is less than a distance between the first light source and the second light source.

2. A measurement apparatus comprising:
a first light emitter including
a first light source that emits first emitted light,
a second light source that is adjacent to the first light source, and emits second emitted light, and
a first sub-mount that supports the first light source and the second light source;
a diffuser disposed in an optical path of each of the first emitted light and the second emitted light;
a photodetector that detects first reflected light and second reflected light, the first reflected light emanating from a subject due to the first emitted light diffused by the diffuser, the second reflected light emanating from the subject due to the second emitted light diffused by the diffuser; and
a processing circuit that, based on a result of detection of the first reflected light and the second reflected light by the photodetector, generates and outputs information related to the subject.

3. The measurement apparatus according to claim 2,
wherein a distance on the diffuser between a center of the first light spot and a center of the second light spot is less than or equal to 5 mm.

4. The measurement apparatus according to claim 2,
wherein the first emitted light and the second emitted light are incident on the diffuser in at least partially overlapping relation to each other.

5. The measurement apparatus according to claim 2, further comprising:
a first collimator lens disposed in an optical path between the first light source and the diffuser; and
a second collimator lens disposed in an optical path between the second light source and the diffuser.

6. The measurement apparatus according to claim 2,
wherein the first emitted light is incident on the diffuser at an incident angle equal to an incident angle at which the second emitted light is incident on the diffuser.

7. The measurement apparatus according to claim 2,
wherein the diffuser has depressions or projections on a surface of the diffuser.

8. The measurement apparatus according to claim 2,
wherein the first emitted light has a wavelength of greater than or equal to 650 nm and less than 805 nm, and
wherein the second emitted light has a wavelength of greater than or equal to 805 nm and less than or equal to 950 nm.

9. The measurement apparatus according to claim 2,
wherein the first emitted light has a wavelength equal to a wavelength of the second emitted light.

10. The measurement apparatus according to claim 2, further comprising
a control circuit that controls the first light source, the second light source, and the photodetector,
wherein the first reflected light and the second reflected light are pulsed light;
wherein the control circuit
causes the first light source to emit the first emitted light, and causes the second light source to emit the second emitted light,
causes the photodetector to detect a first component of the first reflected light in a first falling period, the first falling period being a period from when a decrease in intensity of the first emitted light begins to when the decrease ends, and
causes the photodetector to detect a second component of the second reflected light in a second falling period, the second falling period being a period from when a decrease in intensity of the second emitted light begins to when the decrease ends, and
wherein the processing circuit generates the information, based on an intensity of the first component of the first reflected light detected by the photodetector and an intensity of the second component of the second reflected light detected by the photodetector.

11. The measurement apparatus according to claim 2, further comprising
a third light source that emits third emitted light,
wherein the photodetector further detects third reflected light emanating from the subject due to the third emitted light diffused by the diffuser, and
wherein the processing circuit generates and outputs the information, based on a result of detection of the first reflected light, the second reflected light, and the third reflected light by the photodetector.

12. The measurement apparatus according to claim 2,
wherein the first light emitter further includes:
a third light source that emits third emitted light;
a fourth light source that emits fourth emitted light;
a second sub-mount that supports the third light source and the fourth light source; and
a housing that houses the first light source, the second light source, the third light source, and the fourth light source,
wherein the photodetector further detects third reflected light and fourth reflected light, the third reflected light emanating from the subject due to the third emitted light diffused by the diffuser, the fourth reflected light emanating from the subject due to the fourth emitted light diffused by the diffuser, and
wherein the processing circuit generates and outputs the information, based on a result of detection of the first reflected light, the second reflected light, the third reflected light, and the fourth reflected light by the photodetector.

13. The measurement apparatus according to claim 2,
wherein the processing circuit
estimates a distance from the photodetector to at least one measurement point on the subject, based on the result of detection by the photodetector;
estimates a position of the at least one measurement point, based on the distance from the photodetector to the at least one measurement point on the subject that has been estimated by the processing circuit;
estimates a first incident angle and a second incident angle, based on the position estimated by the processing circuit, the first incident angle being an angle at which the first emitted light is incident on the at least one measurement point, the second incident angle being an angle at which the second emitted light is incident on the at least one measurement point;
corrects a signal representative of the result of detection, based on the first incident angle estimated by the processing circuit, the second incident angle estimated by the processing circuit, and previously generated calibration data, the calibration data defining a relationship between an incident angle of light on the subject and a reflectance of the subject to the light; and
generates the information, based on the signal corrected by the processing circuit.

14. The measurement apparatus according to claim 2,
wherein the subject is a living body, and
wherein the information includes at least one selected from the group consisting of blood flow, oxygen saturation, pulse rate, and blood pressure.

15. The measurement apparatus according to claim 2,
wherein the subject is a living body, and
wherein the information includes information representative of brain blood flow of the living body.

16. The measurement apparatus according to claim 2, further comprising:
a second light emitter including
a third light source that emits third emitted light,
a fourth light source that is adjacent to the third light source, and emits fourth emitted light, and
a second sub-mount that supports the third light source and the fourth light source; and
a mirror that causes the first emitted light, the second emitted light, the third emitted light, and the fourth emitted light to be concentrated and incident on one region of the diffuser, by changing a direction of propagation of at least one selected from the group consisting of light emitted from the first light emitter and light emitted from the second light emitter.

17. A measurement apparatus comprising:
a light source;
a diffuser disposed in an optical path of emitted light emitted from the light source;
a photodetector that detects reflected light, the reflected light emanating from a subject due to the emitted light diffused by the diffuser; and
a processing circuit that, based on a result of detection of the reflected light by the photodetector, generates and outputs information related to the subject,
wherein the processing circuit
estimates a distance from the photodetector to at least one measurement point on the subject, based on the result of detection by the photodetector,
estimates a position of the at least one measurement point, based on the distance estimated by the processing circuit,
estimates an incident angle of the emitted light on the at least one measurement point, based on the position estimated by the processing circuit,
corrects a signal representative of the result of detection, based on the incident angle estimated by the processing circuit, and previously generated calibration data, the calibration data defining a relationship between an incident angle of the emitted light on the subject and a reflectance of the subject to the emitted light, and
generates the information, based on the signal corrected by the processing circuit.

18. A method comprising:
executing a set of operations multiple times under multiple conditions, the set of operations including
emitting light from a light source toward a subject or a plate to measure a reflectance for at least one measurement point on the subject or on the plate, the plate being used for calibration and similar in optical characteristics to the subject, and
estimating an incident angle of the light on the at least one measurement point,
the multiple conditions comprising varying a distance between the subject or the plate and the light source; and
generating calibration data, the calibration data defining a relationship between the incident angle of the light on the at least one measurement point, and the reflectance.
